(19) United States
(12) Patent Application Publication
(10) Pub. No.: US 2024/0067665 A1
(43) Pub. Date: Feb. 29, 2024

(54) SMALL MOLECULES FOR BORON NEUTRON CAPTURE THERAPY

(71) Applicant: Aviko Radiopharmaceuticals, LLC, New York, NY (US)

(72) Inventors: Andrew William Stamford, Chatham, NJ (US); Zhuming Zhang, HIllsborough, NJ (US)

(21) Appl. No.: 18/232,586

(22) Filed: Aug. 10, 2023

Related U.S. Application Data
(60) Provisional application No. 63/397,626, filed on Aug. 12, 2022.

(51) Int. Cl. C07F 5/02; A61K 41/00

(52) U.S. Cl. CPC C07F 5/022; A61K 41/0095; C07B 2200/05; C07B 2200/07

(57) ABSTRACT

Disclosed are compounds, compositions, and methods useful for boron neutron capture therapy.

### RELATED APPLICATION

This application claims the benefit of priority to U.S. Provisional Patent Application No. 63/397,626, filed Aug. 12, 2022.

### BACKGROUND

Boron neutron capture therapy (BNCT) is a bimodal cancer therapy that requires a boron-containing molecule that selectively partitions into cancer cells and an external neutron beam directed to the cancer. Capture of thermal neutrons by the ¹⁰B nuclei inside the cancer cells results in nuclear fission to give high-energy alpha particles and recoiling ⁷Li nuclei. The high-energy particles damage the tumor cells resulting in tumor cell death while sparing surrounding healthy tissue. New boron delivery agents that selectively partition into tumor tissue in combination with an external neutron beam directed to the tumor may be useful in boron neutron capture therapy for the treatment of various solid tumors.

4-Borono-L-phenylalanine (BPA) is approved in Japan for use in combination with an external neutron beam device for the treatment of recurrent unresectable head & neck cancer. While BPA has demonstrated therapeutic utility in boron neutron capture therapy, it has limitations. Its selectivity for tumor tissue over healthy tissue and its tumor uptake are considered to meet only the minimal requirements for a successful boron neutron capture therapy agent. Agents that have improved uptake into tumor cells and have an improved tumor:healthy tissue ratio relative to BPA may result in improved anti-cancer efficacy.

### SUMMARY

One aspect of the invention provides compounds, compositions, and methods useful for boron neutron capture therapy.

Accordingly, provided herein is a compound having the structure of Formula (I):

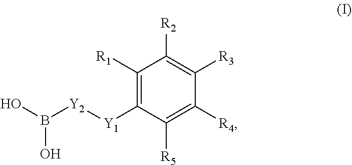

wherein
Y₁ is absent or —O—;
Y₁ is optionally substituted —alkylene—;
R₁, R₂, and R₅ are each independently selected from —H and halo;
R₃ is selected from —H, halo, and —X₁-X₂;
R₄ is selected from —H, halo, and —X₁-X₂;
X₁ is —alkylene—;
X₂ is —C(H)(NH₂)CO₂H; and
the compound is racemic, enriched in one enantiomer, or a single enantiomer;
provided that the compound comprises one and only one occurrence of —X₁-X₂;
or a pharmaceutically acceptable salt thereof.

Another aspect of the invention relates to a method of treating cancer, comprising: i) administering to a subject in need thereof a compound of Formula (I) or pharmaceutical composition comprising a compound of Formula (I), wherein the compound accumulates in a plurality of cancer cells in the subject; and ii) irradiating the plurality of cancer cells with neutrons.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the detailed description, and from the claims.

### DETAILED DESCRIPTION

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions of the present invention may exist in particular geometric or stereoisomeric forms. In addition, polymers of the present invention may also be optically active. The present invention contemplates all such compounds, including cis-and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

“Geometric isomer” means isomers that differ in the orientation of substituent atoms in relationship to a carbon-carbon double bond, to a cycloalkyl ring, or to a bridged bicyclic system. Atoms (other than H) on each side of a carbon- carbon double bond may be in an E (substituents are on opposite sides of the carbon- carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in “atropisomeric” forms or as “atropisomers.” Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from a mixture of isomers. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Percent purity by mole fraction is the ratio of the moles of the enantiomer (or diastereomer) or over the moles of the enantiomer (or diastereomer) plus the moles of its optical isomer. When the stereochemistry of a disclosed compound is named or depicted by structure, the named or depicted stereoisomer is at least about 60%, about 70%, about 80%, about 90%, about 99% or about 99.9% by mole fraction pure relative to the other stereoisomers. When a single enantiomer is named or depicted by structure, the depicted or named enantiomer is at least about 60%, about 70%, about 80%, about 90%, about 99% or about 99.9% by mole fraction pure. When a single diastereomer is named or depicted by structure, the depicted or named diastereomer is at least about 60%, about 70%, about 80%, about 90%, about 99% or about 99.9% by mole fraction pure.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

Structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon, or of a boron with ¹⁰B-enriched boron, are within the scope of this invention.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “pharmaceutically acceptable cocrystals” refers to solid coformers that do not form formal ionic interactions with the small molecule.

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” or “subject” refers to a mammal in need of a particular treatment. In certain embodiments, a patient is a primate, canine, feline, or equine. In certain embodiments, a patient is a human.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C1-C30 for straight chains, C3-C30for branched chains), and more preferably 20 or fewer. Alkyl goups may be substituted or unsubstituted.

As used herein, the term “heteroalkyl” refers to an alkyl moiety as hereinbefore defined which contain one or more oxygen, sulfur, nitrogen, phosphorus, or silicon atoms in place of carbon atoms.

As used herein, the term “haloalkyl” refers to an alkyl group as hereinbefore defined substituted with at least one halogen.

As used herein, the term “hydroxyalkyl” refers to an alkyl group as hereinbefore defined substituted with at least one hydroxyl.

As used herein, the term “alkylene” refers to an alkyl group having the specified number of carbons, for example from 1 to 12 carbon atoms, that contains two points of attachment to the rest of the compound on its longest carbon chain. Non-limiting examples of alkylene groups include methylene —(CH₂)—, ethylene —(CH₂CH₂)—, n-propylene —(CH₂CH₂CH₂)—, isopropylene —(CH₂CH(CH₃))—, and the like. Alkylene groups can be cyclic or acyclic, branched or unbranched carbon chain moiety, and may be optionally substituted with one or more substituents.

“Cycloalkyl” means mono- or bicyclic or bridged or spirocyclic, or polycyclic saturated carbocyclic rings, each having from 3 to 12 carbon atoms. Preferred cycloalkyls have from 3-10 carbon atoms in their ring structure, and more preferably have 3-6 carbons in the ring structure. Cycloalkyl groups may be substituted or unsubstituted.

As used herein, the term “halocycloalkyl” refers to a cycloalkyl group as hereinbefore defined substituted with at least one halogen.

“Cycloheteroalkyl” refers to a cycloalkyl moiety as hereinbefore defined which contain one or more oxygen, sulfur, nitrogen, phosphorus, or silicon atoms in place of carbon atoms. Preferred cycloheteroalkyls have from 4-8 carbon atoms and heteroatoms in their ring structure, and more preferably have 4-6 carbons and heteroatoms in the ring structure. Cycloheteroalkyl groups may be substituted or unsubstituted.

Unless the number of carbons is otherwise specified, “lower alkyl,” as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In certain embodiments, a substituent designated herein as alkyl is a lower alkyl.

“Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

“Alkynyl” refers to hydrocarbyl moieties of the scope of alkenyl, but having one or more triple bonds in the moiety.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). Preferably, aryl groups include 5- to 12-membered rings, more preferably 6- to 10-membered rings The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carboycyclic aryl groups include benzene, naphthalene, phenanthrene, phenol, aniline, and the like. Heteroaryl groups include substituted or unsubstituted aromatic 3- to 12-membered ring structures, more preferably 5- to 12-membered rings, more preferably 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like. Aryl and heteroaryl can be monocyclic, bicyclic, or polycyclic.

The term “halo”, “halide”, or “halogen” as used herein means halogen and includes, for example, and without being limited thereto, fluoro, chloro, bromo, iodo and the like, in both radioactive and non-radioactive forms. In a preferred embodiment, halo is selected from the group consisting of fluoro, chloro and bromo.

The terms “heterocyclyl” or “heterocyclic group” refer to 3- to 12-membered ring structures, more preferably 5- to 12-membered rings, more preferably 5- to 10-membered rings, whose ring structures include one to four heteroatoms. Heterocycles can be monocyclic, bicyclic, spirocyclic, or polycyclic. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C1_6 alkyl, C3-6 cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, the definition of each expression, e.g., alkyl, m, n, etc., when it occurs more than once in any structure, is intended to be independent of its definition elsewhere in the same structure.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, a “small molecule” refers to an organic, inorganic, or organometallic compound typically having a molecular weight of less than about 1000. In some embodiments, a small molecule is an organic compound, with a size on the order of 1 nm. In some embodiments, small molecule drugs of the invention encompass oligopeptides and other biomolecules having a molecular weight of less than about 1000.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments. A series of treatments may comprise a second or subsequent treatment weeks to months after a first or preceding treatment.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter ascompared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation and down-regulation, e.g., enhancing or inhibiting a response.

A “radiopharmaceutical agent,” as defined herein, refers to a pharmaceutical agent which contains at least one radiation-emitting radioisotope. Radiopharmaceutical agents are routinely used in nuclear medicine for the diagnosis and/or therapy of various diseases. The radiolabelled pharmaceutical agent, for example, a radiolabelled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

For purposes of this invention, the chemical elements are identified in accordance with the Periodic Table of the Elements, CAS version, Handbook of Chemistry and Physics, 67th Ed., 1986-87, inside cover.

### Compounds of the Invention

One aspect of the invention relates to a compound of Formula (I):

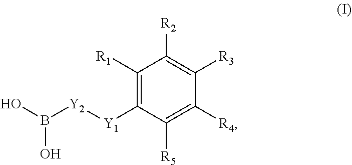

wherein
Y₁ is absent or —O—;
Y₂ is optionally substituted —alkylene—;
R₁, R₂, and R₅ are each independently selected from —H and halo;
R₃ is selected from —H, halo, and —X₁-X₂;
R₄ is selected from —H, halo, and —X₁-X₂;
X₁ is —alkylene—;
X₂ is —C(H)(NH₂)CO₂H; and
the compound is racemic, enriched in one enantiomer, or a single enantiomer;
provided that the compound comprises one and only one occurrence of —X₁-X₂; or a pharmaceutically acceptable salt thereof.

In certain embodiments, the compound having the structure of Formula (IA):

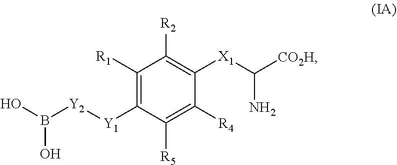

wherein R₄ is selected from —H and halo.

In certain embodiments, each of R₁, R₂, R₄, and R₅ is —H.

In certain embodiments, one of R₁, R₂, R₄, and R₅ is halo, and each of the remainder of R₁, R₂, R₄, and R₅ is —H.

In certain embodiments, the halo is —F.

In certain embodiments, the compound having the structure selected from:

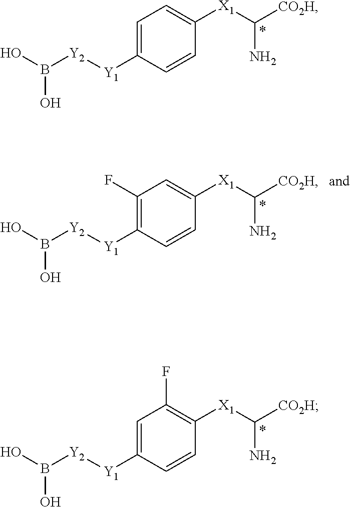

wherein * indicates a chiral carbon with an absolute configuration of (S) or (R); and the compound is not racemic.

In certain embodiments, the absolute configuration of the chiral carbon is (S).

In certain embodiments, the absolute configuration of the chiral carbon is (R).

In certain embodiments, the compound having the structure of Formula (TB):

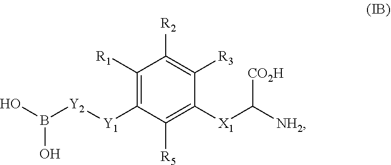

wherein R₃ is selected from —H and halo.

In certain embodiments, each of R₁, R₂, R₃, and R₅ is —H.

In certain embodiments, one of R₁, R₂, R₃, and R₅ is halo, and each of the remainder of R₁, R₂, R₃, and R₅ is hydrogen.

In certain embodiments, the halo is —F.

In certain embodiments, the compound having the structure selected from:

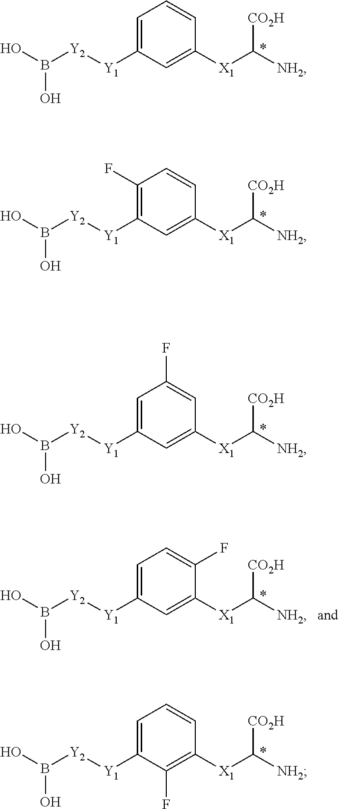

wherein * indicates a chiral carbon with an absolute configuration of (S) or (R); and the compound is not racemic.

In certain embodiments, the absolute configuration of the chiral carbon is (S).

In certain embodiments, the absolute configuration of the chiral carbon is (R).

In certain embodiments, X₁ is —(C₁-C₄)alkylene—.

In certain embodiments, X₁ is —CH₂—.

In certain embodiments, Y₁ is —O—.

In certain embodiments, Y₂ is unsubstituted —(C₁C₄)alkylene—.

In certain embodiments, Y₂ is selected from —CH₂— and —CH₂CH₂—.

In certain embodiments, Y₂ is substituted —(C₁C₄)alkylene—.

In certain embodiments, Y₂ is substituted with a halo, alkyl, heteroalkyl, cycloalkyl, or cyclohetero alkyl.

In certain embodiments, Y₂ is selected from —C(Y₃)(Y₄)— and —C(Y₃)(Y₄)CH₂—; and Y₃ and Y₄ are each independently selected from —H, halo, alkyl, and heteroalkyl, provided that at least one of Y₃ and Y₄ is not —H; or Y₃ and Y₄ taken together with the carbon to which they are bonded form a cycloalkyl, cycloheteroalkyl, spiro cycloalkyl or a spiro cycloheteroalkyl.

In certain embodiments, Y₃ and Y₄ taken together with the carbon to which they are bonded form a cyclopropyl.

In certain embodiments, Y₁ is absent.

In certain embodiments, Y₂ is unsubstituted —(C₁-C₄)alkylene—.

In certain embodiments, Y₂ is selected from —CH₂— and —CH₂CH₂—.

In certain embodiments, Y₂ is substituted —(C₁-C₄)alkylene—.

In certain embodiments, Y₂ is substituted with a halo, alkyl, heteroalkyl, cycloalkyl, or cycloheteroalkyl.

In certain embodiments, Y₂ is selected from —C(Y₃)(Y₄)— and —C(Y₃)(Y₄)CH₂—; and Y₃ and Y₄ are each independently selected from —H, halo, alkyl, and heteroalkyl, provided that at least one of Y₃ and Y₄ is not —H; or Y₃ and Y₄ taken together with the carbon to which they are bonded form a cycloalkyl, cycloheteroalkyl, spiro cycloalkyl or spiro cycloheteroalkyl.

In certain embodiments, Y₃ and Y₄ taken together with the carbon to which they are bonded form a cyclopropyl.

In certain embodiments, the compound having the structure selected from:

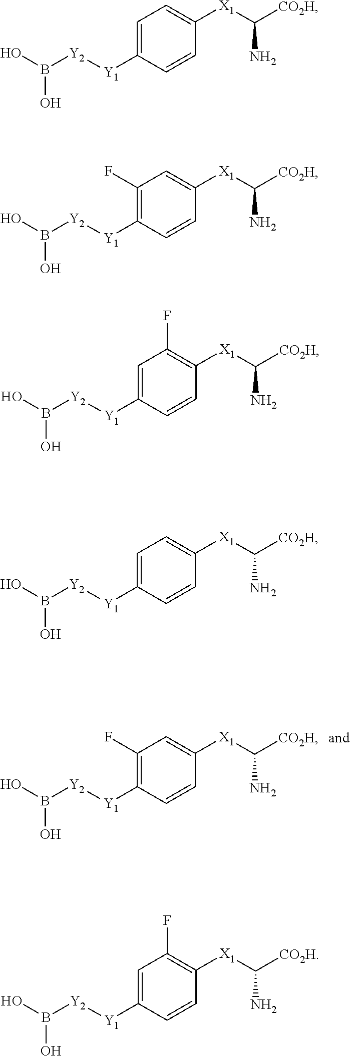

In certain embodiments, the compound having the structure selected from:

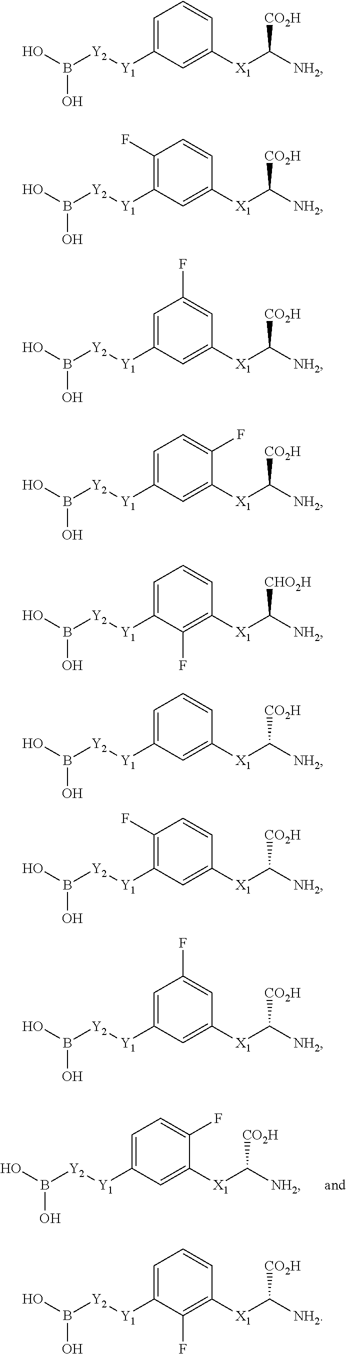

In certain embodiments, the compound having the structure selected from:

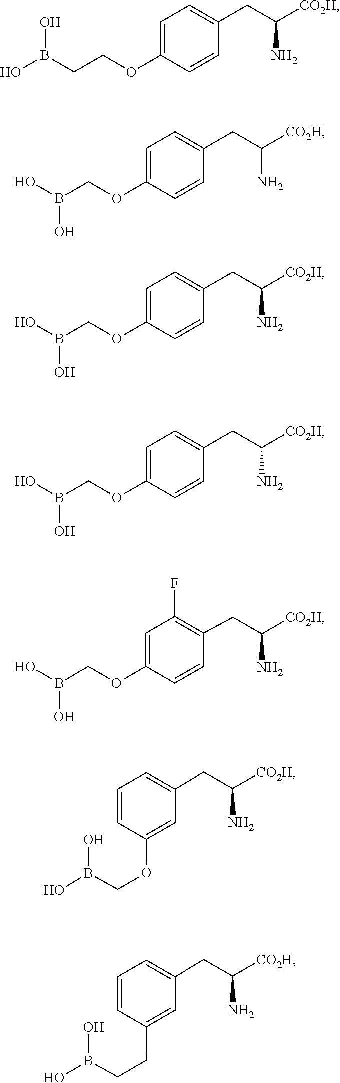

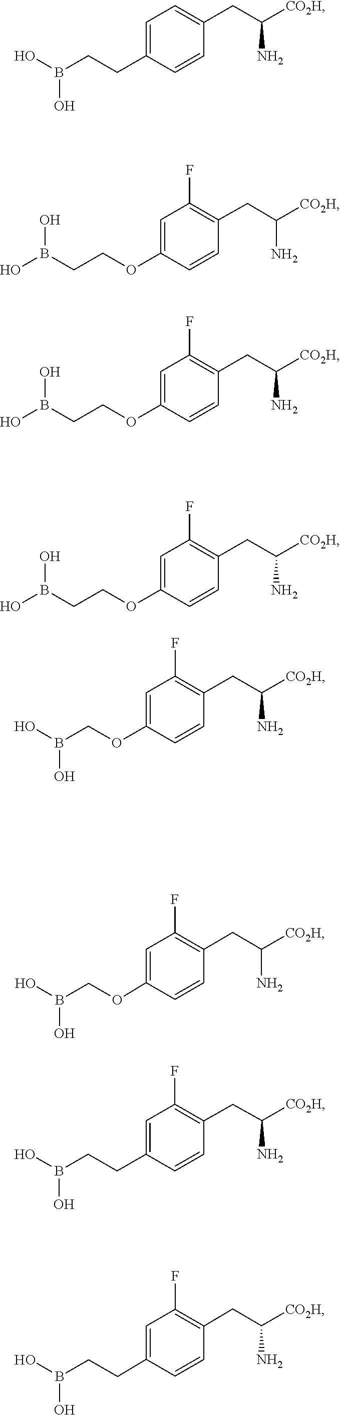

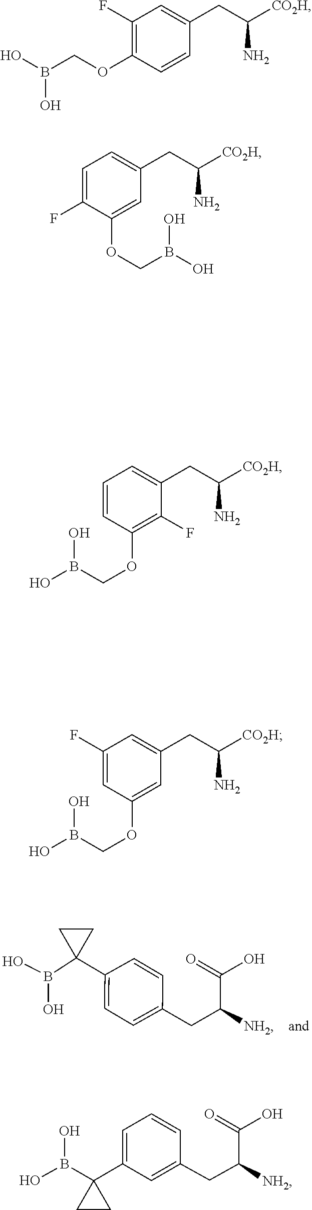 [SMILES: N[C@@H](Cc1ccc(OCB(O)O)c(F)c1)C(=O)O ; N[C@@H](Cc1ccc(F)c(OCB(O)O)c1)C(=O)O ; N[C@@H](Cc1cccc(OCB(O)O)c1F)C(=O)O ; N[C@@H](Cc1cc(F)cc(OCB(O)O)c1)C(=O)O ; N[C@@H](Cc1ccc(C2(B(O)O)CC2)cc1)C(=O)O ; N[C@@H](Cc1cccc(C2(B(O)O)CC2)c1)C(=O)O]

or a pharmaceutically acceptable salt thereof.

In certain embodiments, the compound having the structure:

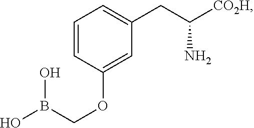 [SMILES: N[C@H](Cc1cccc(OCB(O)O)c1)C(=O)O]

or a pharmaceutically acceptable salt thereof.

In certain embodiments, the boron atom in the compound is ¹⁰B.

In certain embodiments, the compounds are atropisomers. Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention. Such compounds are useful, for example, as analytical tools, as probes in biological assays, or as therapeutic agents in accordance with the present invention. For example, in the case of variable R¹, the (C₁-C₄)alkyl or the —O—(C₁-C₄)alkyl can be suitably deuterated (e.g., —CD₃, —OCD₃).

Also within the scope of the invention are compounds produced comprising the natural distribution of ¹¹B and ¹⁰B.

Also within the scope of the invention are compounds enriched in ¹⁰B, i.e., wherein the is ¹⁰B present in abundance of >20%.

Any compound of the invention can also be radiolabed for the preparation of a radiopharmaceutical agent.

### Methods of Treatment

One aspect of the invention provides compounds, compositions, and methods useful for boron neutron capture therapy.

Another aspect of the invention relates to a method of treating cancer, comprising:
i) administering to a subject in need thereof a compound of Formula (I) or a pharmaceutical composition comprising a compound of Formula (I), wherein the compound accumulates in a plurality of cancer cells in the subject; and
ii) irradiating the plurality of cancer cells with neutrons.

In certain embodiments, the compound selectively or preferentially accumulates in the plurality of cancer cells relative to noncancerous cells in the subject.

In certain embodiments, the irradiation results in conversion of a ¹⁰B atom in the compound to an α-particle and a lithium-7 ion.

In certain embodiments, the compound or the composition is administered intravenously.

In certain embodiments, the compound is continually administered during irradiation with neutrons.

In certain embodiments, in step (i) the compound is administered at about 100 mg/kg/h to about 500 mg/kg/h for a first period of time.

In certain embodiments, in step (i) the compound is administered at about 150 mg/kg/h to about 300 mg/kg/h for a first period of time.

In certain embodiments, the first period of time is about 1 hour to about 3 hours. In certain embodiments, the first period of time is about 2 hours.

In certain embodiments, in step (ii) the compound is administered at about 50 mg/kg/h to about 150 mg/kg/h for a second period of time.

In certain embodiments, in step (ii) the compound is administered at about 100 mg/kg/h to about 200 mg/kg/h for a second period of time.

In certain embodiments, the second period of time is about 0.25 hour to about 1.25 hours.

In certain embodiments, the second period of time is about 0.5 to about 1 hours.

In certain embodiments, the cancer is a solid tumor.

In certain embodiments, the cancer is selected from head and neck cancer, glioblastoma, melanoma, sarcoma, breast cancer, meningioma, lung cancer, mesothelioma, hepatocellular carcinoma, extramammary Paget disease.

In certain embodiments, the cancer is unresectable head and neck cancer.

In certain embodiments of any one of the disclosed methods, the compound of Formula (I) is defined as:

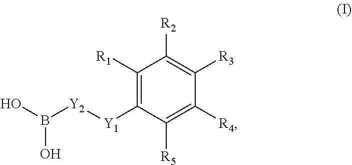

wherein
Y₁ is absent or —O—;
Y₂ is optionally substituted —alkylene—;
R₁, R₂, and R₅ are each independently selected from —H and halo;
R₃ is selected from —H, halo, and —X₁-X₂;
R₄ is selected from —H, halo, and —X₁-X₂;
X₁ is —alkylene—;
X₂ is —C(H)(NH₂)CO₂H; and
the compound is racemic, enriched in one enantiomer, or a single enantiomer;
provided that the compound comprises one and only one occurrence of —X₁-X₂;
or a pharmaceutically acceptable salt thereof.

### Pharmaceutical Compositions, Routes of Administration, and Dosing

In certain embodiments, the invention is directed to a pharmaceutical composition, comprising a compound of the invention and a pharmaceutically acceptable carrier. In certain embodiments, the pharmaceutical composition comprises a plurality of compounds of the invention and a pharmaceutically acceptable carrier.

In certain embodiments, a pharmaceutical composition of the invention further comprises at least one additional pharmaceutically active agent other than a compound of the invention.

Pharmaceutical compositions of the invention can be prepared by combining one or more compounds of the invention with a pharmaceutically acceptable carrier and, optionally, one or more additional pharmaceutically active agents.

In certain embodiments, the pharmaceutical composition further comprises a saccharide.

In certain embodiments, the pharmaceutical composition further comprises a polyhydroxy acid.

In certain embodiments, the pharmaceutical composition further comprises a sugar alcohol.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

In certain embodiments, intravenous administration of a compound may typically be from about 300 mg/kg/day to about 1000 mg/kg/day. In one embodiment, intravenous administration of a compound may typically be from about 400 mg/kg/day to about 600 mg/kg/day. In one embodiment, intravenous administration of a compound may typically be from about 450 mg/kg/day to about 500 mg/kg/day.

Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode that delivers the compound to the desired surface. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound of the invention can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

The invention is further described in the following examples, which do not limit the scope of the invention described in the claims.

Abbreviations used in the instant specification, particularly the schemes and examples, are as follows in Table A:

TABLE A

| Abbreviation | Name |
|---|---|
| ACN or MeCN | acetonitrile |
| AcOH | glacial acetic acid |
| aq | aqueous |
| conc. | concentrated |
| Cu(OAc)₂ | copper(II) acetate |
| DCM | dichloromethane |
| DIEA | diisopropyl-ethyl amine |
| DHP | 3,4-Dihydropyran |
| DMA | dimethylaniline |
| DMAP | 4-dimethylaminopyridine |
| DMF | N,N-dimethylformamide |
| DMSO | dimethylsulfoxide |
| Dppf or DPPF | 1,1′-Bis(diphenylphosphino)ferrocene |
| EtOAc or EA | ethyl acetate |
| EtOH | ethanol |
| GCMS | gas chromatography-mass spectrometry |
| h or hr(s) | hour or hours |
| HPLC | high performance liquid chromatography |
| MeOH | methanol |
| MHz | megahertz |
| min | minute or minutes |
| LCMS | Liquid chromatography mass spectrometry |
| NBS | N-Bromosuccinimide |
| NIS | N-iodosuccinimide |
| NMR | nuclear magnetic resonance |
| Pd(PPh₃)₄ | tetrakis(triphenylphosphine)palladium |
| Pd(OAc)₂ | palladium (II) acetate |
| PE | petrolum ether |
| PPh₃ | triphenylphosphine |
| Pd(dppf)Cl₂ | 1,1′-bis(diphenylphosphino)ferrocene- |
| | palladium(II)dichloride |
| FCC | Flash column chromatography |
| rt or RT | room temperature |
| TBS | tert-Butyldimethylsilyl |
| TEA or Et₃N | triethylamine |
| TFA | trifluoroacetic acid |
| THF | tetrahydrofuran |
| TLC | thin layer chromatography |
| PTSA | p-Toluenesulfonic acid |

### Example 1. Preparation of Compounds

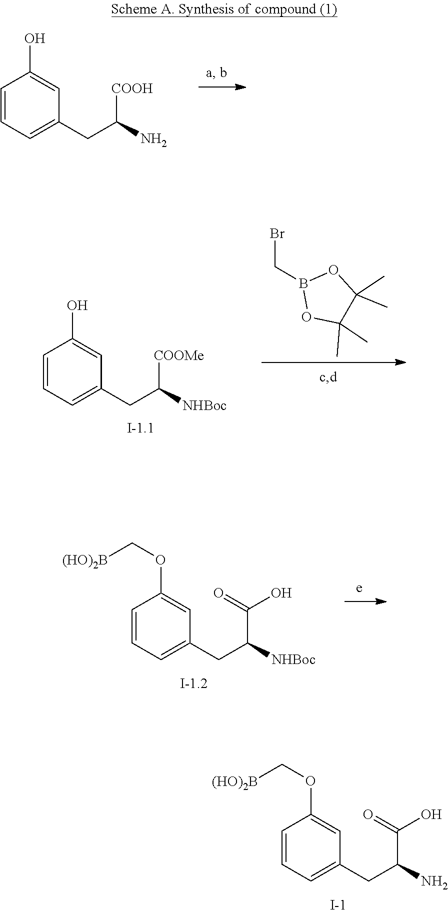 [SMILES: N[C@@H](Cc1cccc(O)c1)C(=O)O ; COC(=O)[C@H](Cc1cccc(O)c1)NC(=O)OC(C)(C)C ; CC1(C)OB(CBr)OC1(C)C ; CC(C)(C)OC(=O)N[C@@H](Cc1cccc(OCB(O)O)c1)C(=O)O ; N[C@@H](Cc1cccc(OCB(O)O)c1)C(=O)O ; [I-]]

Reagents and Conditions: (a) SOCl₂, MeOH, 70° C.; (b) Boc₂O, NaHCO₃, dioxane, water, rt; (c) K₂CO₃, DMF, rt; (d) LiOH·H₂O, THF, water, rt; (e) HCl, EA, rt;

### Preparation of methyl (2S)-2-[(tert-butoxycarbonyl)amino]-3-(3-hydroxyphenyl)propanoate (I-1.1)

Step A. To a mixture of 3-hydroxy-L-phenylalanine (5 g, 27.6 mmol) in MeOH (100 mL) at 0° C. was added SOCl₂ (6.57 g, 55 mmol, 2 equiv) dropwise. The reaction mixture was heated at 70° C. for 4 h. The mixture was cooled to room temperature and concentrated under vacuum to give crude methyl (2S)-2-amino-3-(3-hydroxyphenyl)propanoate as a gum, which was used directly in the next step.

Step B. To a mixture of methyl (2S)-2-amino-3-(3-hydroxyphenyl)propanoate (1.37 g, 7.02 mmol, 1 equiv) and NaHCO₃ (1.47 g, 17.5 mmol, 2.5 equiv) in dioxane (15 mL)/water (15 mL) was added di-tert-butyl dicarbonate (1.84 g, 8.4 mmol, 1.2 equiv) at room temperature. The reaction mixture was stirred overnight at room temperature. The resulting mixture was extracted with EtOAc (3×50 mL). The combined organic layers were washed with brine (100 mL), dried over anhydrous Na₂SO₄, filtered, and concentrated. The residue was purified by silica gel column chromatography, eluted with PE/EA (1:2) to afford methyl (2S)-2-[(tert-butoxycarbonyl)amino]-3-(3-hydroxyphenyl)propanoate as a colorless liquid (2.02 g, 99%). LCMS (ESI): mass calcd. for C₁₅H₂₁NO₅, 295.1; m/z found, 196.3 [M+H−Boc]⁺.

### Preparation of (S)-3-(3-(boronomethoxy)phenyl)-2-((tert-butoxycarbonyl)amino)propanoic acid (I-1.2)

Step C. To a mixture of methyl (2S)-2-[(tert-butoxycarbonyl)amino]-3-(3-hydroxyphenyl) propanoate (1 g, 3.39 mmol, 1 equiv) and K₂CO₃ (0.94 g, 6.772 mmol, 2 equiv) in DMF (10 mL) was added 2-(bromomethyl)-4,4,5,5-tetramethyl-1,3,2-dioxaborolane (1.96 g, 6.77 mmol, 2 equiv) dropwise. The reaction mixture was stirred at room temperature overnight, and then diluted with brine (40 mL). The mixture was extracted with EtOAc (5×20 mL). The combined organic layers were washed with brine (30 mL), dried over anhydrous Na₂SO₄, filtered, and concentrated. The crude product (1.8 g) was used in the next step directly without further purification. Note: 2-(iodomethyl)-4,4,5,5-tetramethyl-1,3,2-dioxaborolane was also employed and provided an improved yield.

Step D. To a stirred solution of methyl (25)-2-[(tert-butoxycarbonyl)amino]-3-{3-[(4,4,5,5-tetramethyl-1,3,2-dioxaborolan-2-yl)methoxy]phenyl}propanoate (0.9 g, 2.07 mmol, 1 equiv) in THF (10 mL)/ water (5 mL) was added LiOH·H₂O (0.25 g, 6.01 mmol, 3 equiv) at room temperature. The reaction mixture was stirred at room temperature overnight. The resulting mixture was extracted with EtOAc (3×10 mL). The afforded aqueous layer was acidified to “pH” 5 with 1N HCl (aq.), the resulting mixture was extracted again with EtOAc (5×20 mL). The combined organic layers were washed with brine (30 mL), dried over anhydrous Na₂SO₄, filtered, and concentrated. This resulted in (S)-3-(3-(boronomethoxy)phenyl)-2-((tert-butoxycarbonyl)amino)propanoic acid as a yellow oil (0.65 g, crude). LCMS (ESI): mass calcd. for C₁₅H₂₂BN07, 339.1; m/z found, 240.0 [M+H−Boc]⁺.

### Preparation of (S)-2-amino-3-(3-(boronomethoxy)phenyl)propanoic acid; trifluoroacetic acid salt (I-1)

To a stirred solution of (S)-3-(3-(boronomethoxy)phenyl)-2-((tert-butoxycarbonyl)amino) propanoic acid (0.65 g, crude) in EtOAc (6 mL) was added 4M HCl in EtOAc (3 mL) at room temperature. The resulting mixture was stirred at room temperature overnight. Te resulting mixture was concentrated under reduced pressure. The crude product was purified by Prep-HPLC [with the following conditions (Column: Sunfire prep C18 column, 30*150 mm, 5 μm; Mobile Phase A: Water (0.05% TFA), Mobile Phase B: ACN; Flow rate: 60 mL/min; Gradient: 4% B to 11% B in 7 min, 11% B; Wave Length: 254/220 nm; RT1 (min): 4.95)] to afford (S)-2-amino-3-(3-(boronomethoxy)phenyl)propanoic acid; trifluoroacetic acid as a white solid (70 mg, 9.90%). LCMS (ESI): mass calcd. for C₁₀H₁₄BNO₅, 239.1; m/z found, 240.1 [M+H]⁺. ¹H NMR (400 MHz, Deuterium Oxide) δ 7.23 (t, J =8.1 Hz, 1H), 6.91-6.83 (m, 1H), 6.83-6.73 (m, 2H), 4.17-4.00 (m, 1H), 3.70 (s, 2H), 3.19 (dd, J =14.5, 5.4 Hz, 1H), 3.04 (dd, J =14.5, 7.9 Hz, 1H).

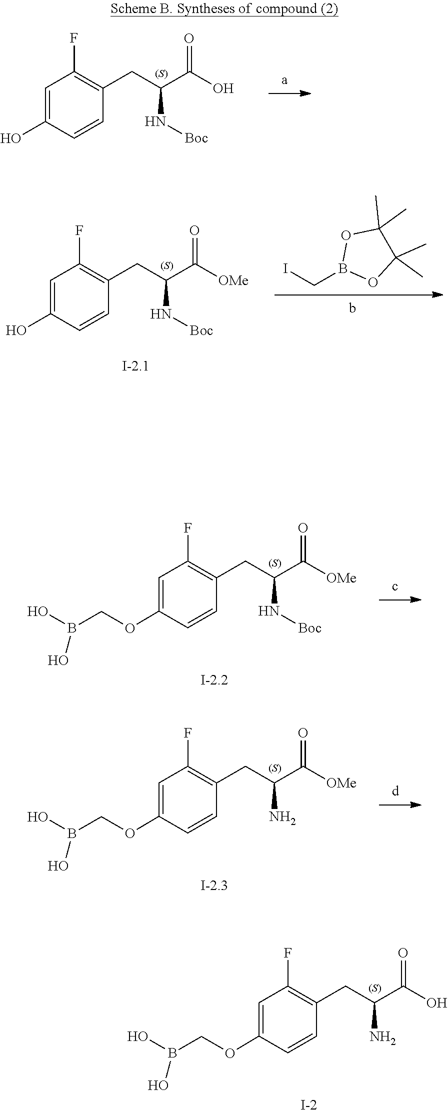

Reagents and Conditions: (a) (Trimethylsilyl)diazomethane (2M in hexanes), MeOH, toluene, 0° C.; (b) K₂CO₃, DMF, rt; (c) HCl (4M in EtOAc), rt; (d) LiOH, THF, H₂O, rt.

### Preparation of methyl (S)-2-((tert-butoxycarbonyl)amino)-3-(2-fluoro-4-hydroxyphenyl) propanoate (I-2.1)

To a stirred mixture of (2S)-2-[(tert-butoxycarbonyl)amino]-3-(2-fluoro-4-hydroxyphenyl)propanoic acid (600 mg, 2.005 mmol, 1 equiv) in toluene (20 mL)/MeOH (5 mL) was added (trimethylsilyl)diazomethane (2M in hexanes) (2.406 mmol, 1.20 mL, 1.2 equiv) at 0° C. under nitrogen atmosphere. The resulting mixture was stirred under nitrogen atmosphere at room temperature for 0.5 h. The reaction was monitored by LCMS. The reaction was quenched by the addition of AcOH (0.5 mL) at room temperature, and then concentrated under reduced pressure. The residue was purified by reverse flash chromatography [with the following conditions: column, C18 silica gel; mobile phase, MeCN in Water (0.1% NH₃·H₂O), 55% to 65% gradient in 10 min; detector, UV 254 nm]. This resulted in methyl (2S)-2-Rtert-butoxycarbonyl)aminol-3-(2-fluoro-4-hydroxyphenyl)propanoate as a white solid (540 mg, 85.97%). LCMS (ESI): mass calcd. for C₁₅H₂₀FNO₅, 313.13; m/z found, 312.0 [M−H]⁻.

### Preparation of methyl (S)-((4-(2-((tert-butoxycarbonyl)amino)-3-methoxy-3-oxopropyl)-3-fluorophenoxy)methyl)boronic acid (I-2.2)

To a stirred mixture of methyl (2S)-2-Rtert-butoxycarbonyl)aminol-3-(2-fluoro-4-hydroxyphenyl)propanoate (540 mg, 1.723 mmol, 1 equiv) and K₂CO₃ (1190.95 mg, 8.615 mmol, 5 equiv) in DMF (10 mL) at room temperature was added 2-(iodomethyl)-4,4,5,5-tetramethyl-1,3,2-dioxaborolane (2308.56 mg, 8.615 mmol, 5 equiv). The resulting mixture was stirred under nitrogen atmosphere at room temperature overnight. The reaction was monitored by LCMS. The resulting mixture was diluted with water, and extracted with EtOAc (3×40 mL). The combined organic layers were washed with brine (2×80 mL), and dried over anhydrous Na2SO4. After filtration, the filtrate was concentrated under reduced pressure. The residue was purified by reverse flash chromatography [with the following conditions: column, C18 silica gel; mobile phase, MeCN in Water (0.1% NH₃·H₂O), 50% to 60% gradient in 10 min; detector, UV 254 nm]. This resulted in (S)-((4-(2-((tert-butoxycarbonyl)amino)-3-methoxy-3-oxopropyl)-3-fluorophenoxy)methyl) boronic acid as a white solid (590 mg, 92.18%). LCMS (ESI): mass calcd. for C₁₆H₂₃BFNO₇, 371.16; m/z found, 272.1 [M+H−Boc]⁺.

### Preparation of 4-[(2S)-2-amino-3-methoxy-3-oxopropyl]-3-fluorophenoxymethylboronic acid (I-2.3)

To a stirred mixture of methyl (S)-((4-(2-((tert-butoxycarbonyl)arnino)-3-methoxy-3-oxopropyl)-3-fluorophenoxy)rnethyl)boronic acid (100 mg, 0.221 mmol, 1 equiv) in EtOAc (2.0 mL) under nitrogen atmosphere at room temperature was added 4 M HCl in EtOAc (2.0 mL) dropwise. The resulting mixture was stirred at room temperature for 5 h, and then concentrated under vacuum. The residue was diluted with water, and basified to “pH” 8 with saturated NaHCO₃ (aq.). The resulting mixture was extracted with EtOAc (2×30 mL). The combined organic layers were washed with brine (2×50 mL), and dried over anhydrous Na₂SO₄. After filtration, the filtrate was concentrated under reduced pressure. The residue was purified by reverse flash chromatography [with the following conditions: column, C18 silica gel; mobile phase, MeCN in Water (0.1% NH₃·H₂O), 20% to 30% gradient in 8 min; detector, UV 254 nm]. This resulted in 4-[(2S)-2-amino-3-methoxy-3-oxopropyl]-3-fluorophenoxymethylboronic acid as a white solid (340 mg, 96.38%). LCMS (ESI): mass calcd. for C₁₁H₁₅BFNO₅, 271.1; m/z found, 272.2 [M+H]⁺.

### Preparation of (S)-2-amino-3-(4-(boronomethoxy)-2-fluorophenyl)propanoic acid; trifluoroacetic acid (I-2)

To a stirred mixture of 4-[(2S)-2-amino-3-methoxy-3-oxopropyl]-3-fluorophenoxymethylboronic acid (340 mg, 1.25 mmol, 1 equiv) in THF (3 mL)/H₂O (1 mL) was added LiOH·H₂O (157.9 mg, 3.76 mmol, 3.00 equiv) at room temperature. The resulting mixture was stirred for 5 h at room temperature under nitrogen atmosphere. The reaction mixture was acidified to pH 6 with 1N HCl (aq.), and then concentrated under reduced pressure. The residue was purified by reverse flash chromatography [with the following conditions: column, C18 silica gel; mobile phase, MeCN in water (0.1% TFA), 10% to 50% gradient in 10 min; detector, UV 254 nm]. The product was further purified by Prep-HPLC [with the following conditions (Column: XBridge Shield RP18 OBD Column, 30*150 mm, 5 μm; Mobile Phase A: water (0.05% TFA), Mobile Phase B: ACN; Flow rate: 60 mL/min; Gradient: 4% B to 10% B in 7 min, 10% B; Wave Length: 254/220 nm; RT1(min): 5.92)]. This resulted in (S)-2-amino-3-(4-(boronomethoxy)-2-fluorophenyl)propanoic acid; trifluoroacetic acid (110 mg, 22.50%) as a white solid. LCMS (ESI): mass calcd. for C₁₀H₁₃BFNO₅, 257.1; m/z found, 257.9 [M+H]⁺, ¹H NMR (400 MHz, Deuterium Oxide) δ 7.17 (t, J=8.6 Hz, 1H), 6.80-6.72 (m, 2H), 4.06 (dd, J=7.5, 5.5 Hz, 1H), 3.75 (s, 2H), 3.24 (dd, J=14.8, 5.6 Hz, 1H), 3.06 (dd, J=14.8, 7.6 Hz, 1H).

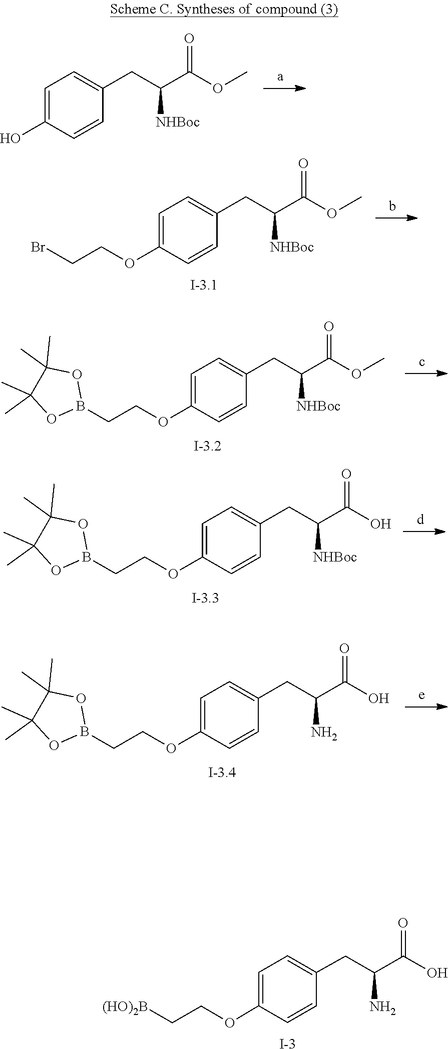

Reagents and Conditions: (a) 1,2-dibromoethane, K₂CO₃, 18-crown-6, 80° C.; (b) CuCl, Xantphos, B₂Pin₂, ^{t}BuOK, DMF, 50° C.; (c) LiOH·H₂O, MeOH, H₂O, rt; (d) HCl, dioxane, rt; (e) NaIO₄, HCl, THF, H₂O, rt.

### Preparation of methyl (S)-3-(4-(2-brornoethoxy)phenyl)-2-((tert-butoxycarbonyl)amino) propanoate (I-3.1)

To a stirred solution of methyl (2S)-2-[(tert-butoxycarbonyl)amino]-3-(4-hydroxyphenyl) propanoate (5 g, 16.930 mmol, 1 equiv) in dibromoethane (20 mL) at room temperature were added K₂CO₃ (16.00 g, 115.801 mmol, 6.84 equiv), 18-crown-6 (0.45 g, 1.693 mmol, 0.1 equiv). The resulting mixture was stirred under nitrogen atmosphere at 80° C. for 24 h. The mixture was allowed to cool down to room temperature and diluted with ethyl acetate (150 mL). Then the mixture was washed with water and brine, dried over anhydrous Na₂SO₄. After filtration, the filtrate was concentrated under reduced pressure. The residue was purified by silica gel column chromatography, eluted with PE/EA (5:1) to afford methyl (S)-3-(4-(2-bromoethoxy)phenyl)-2-((tert-butoxycarbonyl)amino)propanoate as a white solid (5.6 g, 82.22%). LCMS (ESI): mass calcd. for C₁₇H₂₄BrNO₂401.1; m/z found, 302.0 [M-Boc+H]⁺.

### Preparation of methyl (S)-2-((tert-butoxycarbonyl)amino)-3-(4-(2-(4,4,5,5-tetramethyl-1,3,2-dioxaborolan-2-yl)ethoxy)phenyl)propanoate (I-3.2)

To a stirred solution of methyl (S)-3-[4-(2-bromoethoxy)phenyl]-2-[(tert-butoxycarbonyl) amino]propanoate (5.6 g, 13.921 mmol, 1 equiv) and bis(pinacolato)diboron (10.61 g, 41.762 mmol, 3 equiv) in DMF (110 mL) at room temperature were added CuCl (413.44 mg, 4.177 mmol, 0.3 equiv), Xantphos (2.41 g, 4.177 mmol, 0.3 equiv) and Potassium tert-butoxide (1 M in THF) (16.6 mL, 1.2 equiv). The resulting mixture was purged with N2 for 1 min, and then stirred under nitrogen atmosphere at 50° C. for 3 h. The mixture was diluted with EA (80 mL) and washed with water (40 mL) and brine (40 mL). The organic layer was dried over anhydrous Na₂SO₄. After filtration, the filtrate was concentrated under vacuum and the residue was purified by silica gel column chromatography, eluted with PE/EA (4:1) to afford methyl (S)-2-[(tert-butoxycarbonyl)amino]-3-{4-[2-(4,4,5,5-tetramethyl- 1,3,2-dioxaborolan-2-yl)ethoxy]phenyl} propanoate (2.8 g, 44.76%) as a colorless oil. LCMS (ESI): mass calcd. for C₂₃H₃₆BNO₇, 449.3; m/z found, 350.2 [M−Boc+H]⁺.

### Preparation of (S)-2-((tert-butoxycarbonyl)amino)-3-(4-(2-(4,4,5,5-tetramethyl-1,3,2-dioxaborolan-2-yl)ethoxy)phenyl)propanoic acid (I-3.3)

To a stirred solution of methyl (2S)-2-[(tert-butoxycarbonyl)amino]-3-{4-[2-(4,4,5,5-tetramethyl-1,3,2-dioxaborolan-2-yl)ethoxy]phenyl}propanoate (1.08 g, 2.403 mmol, 1 equiv) in H₂O (20 mL) and MeOH (40 mL) at room temperature was added LiOH·H₂O (302.6 mg, 7.21 mmol, 3 equiv) in portions. The resulting mixture was stirred at room temperature for 3 h. Then the mixture was acidified to “pH” 4 with 1N HCl (aq.) and extracted with EA (3×30 mL), dried over anhydrous Na₂SO₄. After filtration, the filtrate was concentrated under reduced pressure. The residue was used in the next step directly without further purification. LCMS (ESI): mass calcd. for C₂₂H₃₄BNO₇, 435.2; m/z found, 336.2 [M−Boc+H]⁺.

### Preparation of (S)-2-amino-3-(4-(2-(4,4,5,5-tetramethyl-1,3,2-dioxaborolan-2-yl)ethoxy)phenyl) propanoic acid (I-3.4)

To a stirred solution of (2S)-2-[(tert-butoxycarbonyl)amino]-3-{4-[2-(4,4,5,5-tetramethyl-1,3,2-dioxaborolan-2-yl)ethoxy]phenyl}propanoic acid (920 mg, 2.11 mmol, 1 equiv) in dioxane (9 mL) was added HCl (gas) solution (9 mL, 4 M in dioxane) dropwise. The resulting mixture was stirred at room temperature for 1 h, and then concentrated under reduced pressure. The residue was used in the next step directly without further purification. LCMS (ESI): mass calcd. for C₁₇H₂₆BNO₅, 335.2; m/z found, 336.2 [M+H]⁺.

### Preparation of (2S)-2-amino-3-{4[2-(dihydroxyboranyl)ethoxy]phenyl}propanoic acid (I-3)

To a stirred solution of (2S)-2-amino-3-{4-[2-(4,4,5,5-tetramethyl-1,3,2-dioxaborolan-2-yl)ethoxy]phenyl}propanoic acid (800 mg, 2.39 mmol, 1 equiv) in THF (20.00 mL) and H₂O (5.00 mL) at room temperature was added sodium periodate (1.53 g, 7.161 mmol, 3 equiv) in portions. The resulting mixture was stirred at room temperature for 5 min, andthen 2N HCl (aq.) (1.00 mL, 2.005 mmol, 0.84 equiv) was added.. The reaction mixture was stirred at room temperature for additional 2 h. After that, the mixture was filtered, and the filter cake was washed with water (2×5 mL). The filtrate was concentrated under reduced pressure. The residue was purified by HP-Flash chromatography [with the following conditions: column, C18 silica gel; mobile phase, MeCN in Water (0.1% FA), 5% to 30% gradient in 20 min; detector, UV 220 nm]. This resulted in (2S)-2-amino-3-{4-[2-(dihydroxyboranyl)ethoxy]phenyl}propanoic acid as a white solid (150 mg, 24.12%). LCMS (ESI): mass calcd. for C₁₁H₁₆BNO₅, 253.1; m/z found, 254.1 [M+H]⁺; ¹H NMR (400 MHz, Deuterium Oxide) δ 7.17 (d, J=8.2 Hz, 2H), 6.92 (d, J=8.2 Hz, 2H), 4.13 (t, J=7.5 Hz, 2H), 3.87 (dd, J=7.6, 5.3 Hz, 1H), 3.14 (dd, J=14.6, 5.2 Hz, 1H), 2.99 (dd, J=14.7, 7.8 Hz, 1H), 1.26 (t, J=7.6 Hz, 2H).

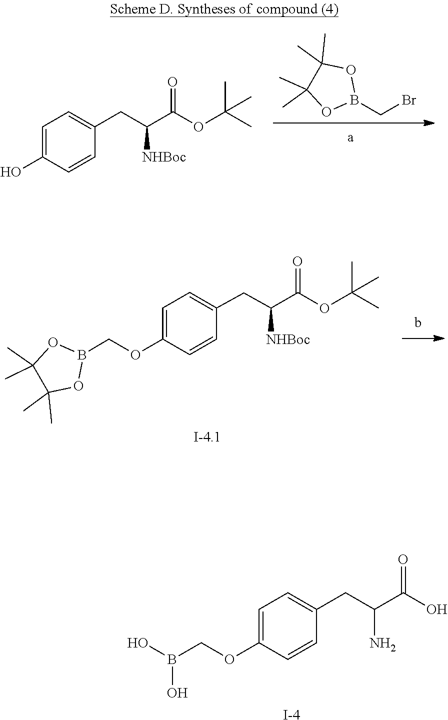

Reagents and Conditions: (a) K₂CO₃, DMF, rt; (b) HCl (4M in EtOAc), EtOAc, rt.

### Preparation of tert-butyl (2S)-2-[(tert-butoxycarbonyl)amino]3-{4-[(4,4,5,5-tetramethyl-1,3,2-dioxaborolan-2-yl)methoxy]phenyl}propanoate (I-4.1)

To a stirred mixture of tert-butyl (2S)-2-[(tert-butoxycarbonyl)amino]-3-(4-hydroxyphenyl) propanoate (1 g, 2.964 mmol, 1 equiv) and K₂CO₃ (0.82 g, 5.928 mmol, 2 equiv) in DMF (10 mL) at room temperature was added 2-(bromomethyl)-4,4,5,5-tetramethyl-1,3,2-dioxaborolane (1.31 g, 5.928 mmol, 2 equiv). The resulting mixture was stirred at room temperature overnight. The resulting mixture was diluted with water, and then extracted with EtOAc (2×200 mL). The combined organic layers were washed with brine (3×300 mL), dried over anhydrous Na₂SO₄. After filtration, the filtrate was concentrated under reduced pressure to afford tert-butyl (2S)-2-[(tert-butoxycarbonyl)amino]-3-{4-[(4,4,5,5-tetramethyl-1,3,2-dioxaborolan-2-yl)methoxy] phenyl}propanoate as a light yellow oil (1.34 g, crude). LCMS (ESI): mass calcd. for C₂₅H₄₀BNO₇, 477.4; m/z found, 378 [M+H−Boc]⁺.

### Preparation of 2-amino-3-{4-[(dihydroxyboranyl)methoxy]phenyl}propanoic acid (I-4)

To a stirred mixture of tert-butyl (2S)-2-[(tert-butoxycarbonyl)amino]-3-{4-[(4,4,5,5-tetramethyl-1,3,2-dioxaborolan-2-yl)methoxy]phenyl}propanoate (670 mg, 1.40 mmol, 1 equiv) in EtOAc (5 mL) at room temperature was added HC₁(g) solution (10 mL, 4M in EtOAc) dropwise. The resulting mixture was stirred at room temperature overnight. The precipitated solids were collected by filtration and washed with ethyl ether (3×5 mL). The crude product (358 mg) was purified by Prep-HPLC with the following conditions (Column: Atlantis Prep T3 OBD Column, 19*150mm, 5 μm; Mobile Phase A: Water (0.1% FA), Mobile Phase B: ACN; Flow rate: 25 mL/min; Gradient: 2% B to 6% B in 6 min, 6% B; Wave Length: 254/220 nm; RT1 (min): 4.57) to afford 2-amino-3-{4-[(dihydroxyboranyl)methoxy]phenyl}propanoic acid as a white solid (49.1 mg, 14.61%).

LCMS (ESI): mass calcd. for C₁₀H₁₄BNO₅, 239.0; m/z found, 240.0 [M+H]⁺. ¹H NMR (400 MHz, Deuterium Oxide) δ 7.17 (d, J=8.7 Hz, 2H), 6.93 (d, J=8.6 Hz, 2H), 3.87 (dd, J=7.8, 5.1 Hz, 1H), 3.75 (s, 2H), 3.15 (dd, J=14.7, 5.2 Hz, 1H), 2.99 (dd, J=14.7, 7.8 Hz, 1H).

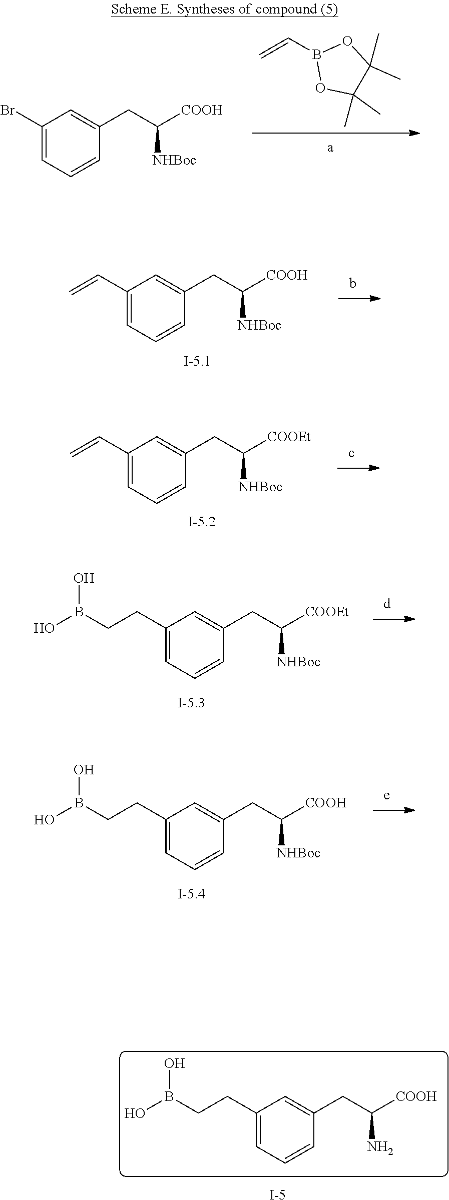

Reagents and Conditions: (a) Pd(OAc)₂, S-phos, K₃PO₄, dioxane, H₂O, 90° C.; (b) EtOH, EDCI, DMAP, DCM, rt; (c) BH3-THF, H₂O, rt; (d) LiOH, MeOH, H₂O, rt; (e) TFA, DCM, rt.

### Preparation of (S)-2-((tert-butoxycarbonyl)amino)-3-(3-vinylphenyl)propanoic acid (I-5.1)

To a stirred mixture of (2S)-3-(3-bromophenyl)-2-[(tert-butoxycarbonyl)amino]propanoic acid (2 g, 5.81 mmol, 1 equiv) and 2-ethenyl-4,4,5,5-tetramethyl-1,3,2-dioxaborolane (1.34 g, 8.72 mmol, 1.5 equiv) in dioxane (50 mL) and H₂O (5 mL) at room temperature was added K₃PO₄ (4.93 g, 23.2 mmol, 4 equiv), S-phos (0.48 g, 1.162 mmol, 0.2 equiv) and Pd(OAc)₂ (0.13 g, 0.581 mmol, 0.1 equiv). The reaction mixture was purged with N₂ for 1 min, and then stirred under nitrogen atmosphere at 90° C. for 8 h. The resulting mixture was concentrated under reduced pressure. The residue was purified by silica gel column chromatography, eluted with PE/EA (3:1) to afford (2S)-2-[(tert-butoxycarbonyl)amino]-3-(3-ethenylphenyl)propanoic acid (2.1 g, crude) as a brown oil. LCMS (ESI): mass calcd. for C₁₆H₂₁NO₄, 291.1; m/z found, 192 [M−Boc+H]⁺.

### Preparation of ethyl (S)-2-((tert-butoxycarbonyl)amino)-3-(3-vinylphenyl)propanoate (I-5.2)

To a stirred mixture of (2S)-2-[(tert-butoxycarbonyl)amino]-3-(3-ethenylphenyl)propanoic acid (2.2 g, 7.551 mmol, 1 equiv), EtOH (3.48 g, 75.510 mmol, 10 equiv) and DMAP (0.09 g, 0.755 mmol, 0.1 equiv) in DCM (60 mL) was at room temperature added EDCI (1.59 g, 8.306 mmol, 1.1 equiv). The reaction mixture was stirred at 25° C. for 8 h, and then concentrated under reduced pressure. The residue was purified by reverse flash chromatography [with the following conditions: column, C18 silica gel; mobile phase, MeCN in Water (0.1% TFA), 10% to 80% gradient in 10 min; detector, UV 254/220 nm] to afford ethyl (2S)-2-[(tert-butoxycarbonyl) amino]-3-(3-ethenylphenyl)propanoate as a light yellow solid (1.5 g, 62.19%). LCMS (ESI): mass calcd. for C₁₈H₂₅NO₄, 319.2; m/z found, 220.1 [M−Boc+H]⁺.

### Preparation of (S)-(3-(2-((tert-butoxycarbonyl)amino)-3-ethoxy-3-oxopropyl)phenethyl) boronic acid (I-5.3)

To a stirred mixture of ethyl (2S)-2-[(tert-butoxycarbonyl)amino]-3-(3-ethenylphenyl) propanoate (800 mg, 2.505 mmol, 1 equiv) in THF (10 mL) at 0° C. was added borane solution (1 M in THF, 5 mL, 5.01 mmol, 2 equiv) dropwise. The reaction mixture was stirred at 25° C. for 2 h, and then quenched by the addition of water at 0° C. The mixture was stirred for additional 2 h at 25° C., and then concentrated under reduced pressure. The residue was purified by reverse flash chromatography [with the following conditions: column, C18 silica gel; mobile phase, MeCN in Water (0.1% TFA), 10% to 50% gradient in 10 min; detector, UV 254/220 nm] to afford 2-{3-[(2S)-2-[(tert-butoxycarbonyl)amino]-3-ethoxy-3-oxopropyl]phenyl} ethylboronic acid as a light yellow oil (600 mg, 65.59%). LCMS (ESI): mass calcd. for C₁₈H₂₈BNO₆, 365.2; m/z found, 266.2 [M−Boc+H]⁺.

### Preparation of (S)-3-(3-(2-boronoethyl)phenyl)-2-((tert-butoxycarbonyl)amino)Propanoic Acid (I-5.4)

To a stirred mixture of 2-{3-[(2S)-2-[(tert-butoxycarbonyl)amino]-3-ethoxy-3-oxopropyl] phenyl}ethylboronic acid (600 mg, 1.64 mmol, 1 equiv) in MeOH (6 mL) and H₂O (6 mL) was added LiOH·H₂O (344.70 mg, 8.215 mmol, 5 equiv). The mixture was stirred at 25° C. for 2 h, and then acidified to “pH” 4 with 2N HCl (aq.). The resulting mixture was concentrated under reduced pressure. The residue was purified by reverse flash chromatography [with the following conditions: column, C18 silica gel; mobile phase, MeCN in Water (0.1% TFA), 10% to 50% gradient in 10 min; detector, UV 254/220 nm] to afford (S)-3-(3-(2-boronoethyl)phenyl)-2-((tert-butoxycarbonyl)amino)propanoic acid as an off-white solid (580 mg, crude). LCMS (ESI): mass calcd. for C₁₆H₂₄BNO₆, 337.2; m/z found, 238.1 [M−Boc+H]⁺.

### Preparation of (S)-2-amino-3-(3-(2-boronoethyl)phenyl)propanoic acid (I-5)

To a stirred mixture of (S)-3-(3-(2-boronoethyl)phenyl)-2-((tert-butoxycarbonyl)amino)propanoic acid (480 mg, 1.42 mmol, 1 equiv) in DCM (10 mL) at room temperature was added TFA (1 mL) dropwise. The mixture was stirred at 25° C. for 2 h, and then concentrated under reduced pressure. The residue was purified by Prep-HPLC [with the following conditions (Column: XBridge Prep Phenyl OBD Column, 19*150 mm, 5 μm; Mobile Phase A: Water (0.1% FA), Mobile Phase B: MeOH-HPLC; Flow rate: 25 mL/min; Gradient: 9% B to 10% B in 6 min, 10% B; Wave Length: 220 nm; RT1(min): 4.11)] to afford (2S)-2-amino-3-{3-[2-(dihydroxyboranyl)ethyl]phenyl}propanoic acid as an off-white solid (83.3 mg, 24.19%). LCMS (ESI): mass calcd. for C₁₁H₁₆BNO₄, 237.1; m/z found, 238.0 [M+H]⁺; ¹H NMR (400 MHz, Methanol-d₄) δ 7.24 (t, J=7.5 Hz, 1H), 7.20-7.03 (m, 3H), 4.00-3.84 (m, 1H), 3.31-3.21 (m, 1H), 3.01 (dd, J=14.5, 8.3 Hz, 1H), 2.67 (t, J=8.1 Hz, 2H), 1.11 (t, J=8.2 Hz, 2H).

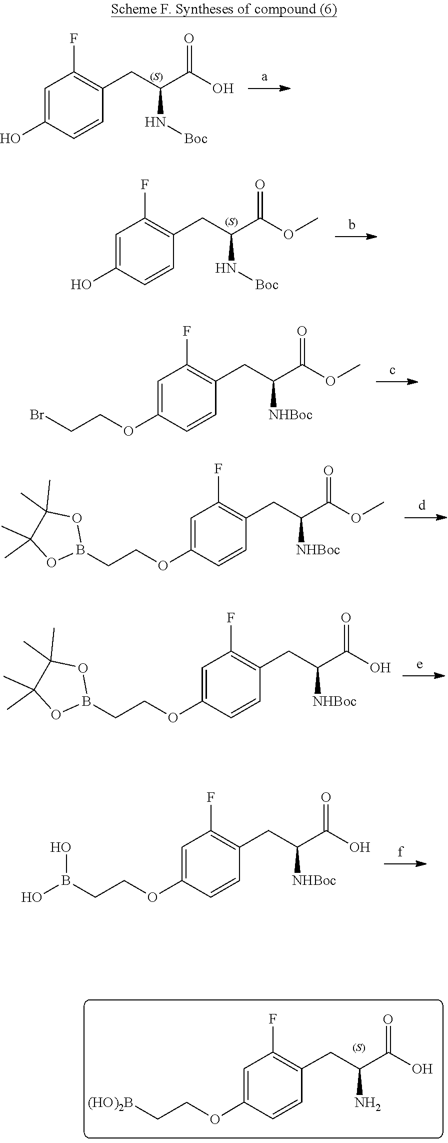

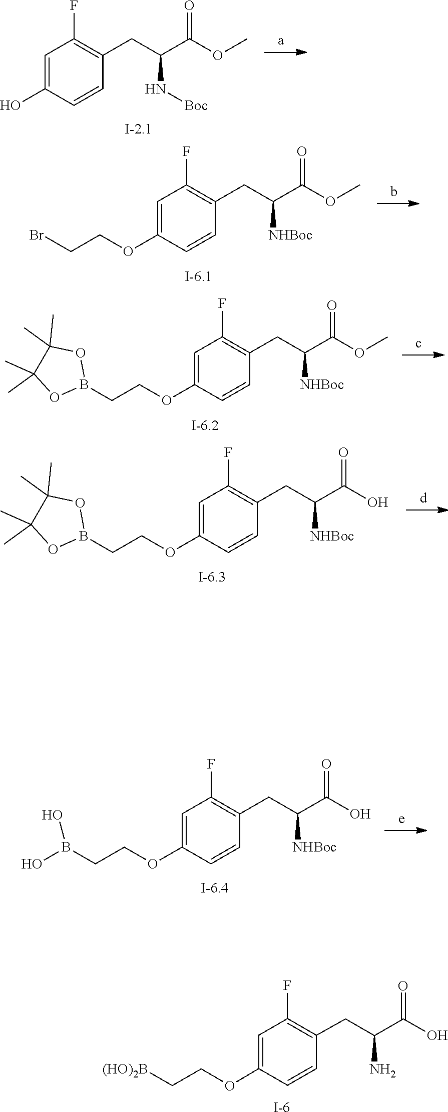

Reagents and Conditions: (a) 1,2-dibromoethane, K₂CO₃, 18-crown-6, 80° C.; (b) CuCl, Xantphos, B₂Pin₂, ^{t}BuOK, DMF, 50° C.; (c) LiOH·H₂O, THF, H₂O, rt; (d) NaIO4, HCl, THF, H₂O, rt; (e) HCl, EA, rt.

### Preparation of Methyl (2S)-3-(4-(2-brornoethoxy)-2-fluorophenyl)-2-((tert-butoxycarbonyl) amino)propanoate (I-6.1)

To a stirred solution of methyl (2S)-2-[(tert-butoxycarbonyl)amino]-3-(2-fluoro-4-hydroxyphenyl)propanoate (I-2.1, 1.03 g, 3.28 mmol, 1 equiv) in 1,2-dibromoethane (27.2 mL) at room temperature were added K₂CO₃ (3.12 g, 22.62 mmol, 6.9 equiv) and 18-crown-6 (86.6 mg, 0.327 mmol, 0.1 equiv). The resulting mixture was stirred under nitrogen atmosphere at 80° C. for 24 h . The reaction was allowed to cool down to room temperature and diluted with ethyl acetate (80 mL). Then the mixture was washed with brine (3×50 mL), dried over anhydrous Na₂SO₄. After filtration, the filtrate was concentrated under reduced pressure. The residue was purified by silica gel column chromatography, eluted with PE/EA (4:1) to afford methyl (2S)-3-[4-(2-bromoethoxy)-2-fluorophenyl]-2-[(tert-butoxycarbonyl)amino]propanoate as a light yellow oil (1.17 g, 85.20%). LCMS (ESI): mass calcd. for C₁₇H₂₃BrNO₅, 419.1; m/z found, 320 [M−Boc+H]⁺.

### Preparation of (2S)-2-[(tert-butoxycarbonyl)amino]-3-{2-fluoro-4-[2-(4,4,5,5-tetramethyl-1,3,2-dioxaborolan-2-yl)ethoxy]phenyl}propanoate (I-6.2)

A mixture of CuCl (82.9 mg, 0.837 mmol, 0.3 equiv) and Xantphos (484.62 mg, 0.837 mmol, 0.3 equiv) in DMF (22 mL) was stirred under nitrogen atmosphere at room temperature for 0.5 h. Next, methyl (2S)-3-[4-(2-bromoethoxy)-2-fluorophenyl]-2-[(tert-butoxycarbonyl)amino] propanoate (1.17 g, 2.79 mmol, 1 equiv), bis(pinacolato)diboron (2.13 g, 8.372 mmol, 3 equiv) and potassium tert-butoxide (1 M in THF) (3.3 mL, 3.3 mmol, 1.2 equiv) were added. The resulting mixture was purged with N₂ for 1 min, and then stirred under nitrogen atmosphere at 50° C. 3 h. The reaction was quenched with addition of water (80 mL) and the mixture was extracted with EA (3×60 mL). The combined organic layers were washed with brine (100 mL) and dried over anhydrous Na₂SO₄. After filtration, the filtrate was concentrated under vacuum and the residue was purified by silica gel column chromatography, eluted with PE/EA (4:1) to afford methyl (2S)-2-[tert-butoxycarbonyl)amino]-3-{2-fluoro-4-[2-(4,4,5,5-tetramethyl-1,3,2-dioxaborolan-2-yl)ethoxy]phenyl}propanoate as a light yellow oil (587 mg, 44.96%). LCMS (ESI): mass calcd. for C₂₃H₃₅BFNO₇, 467.3; m/z found, 368.2 [M−Boc+H]⁺.

### Preparation of (S)-2-((tert-butoxycarbonyl)amino)-3-(2-fluoro-4-(2-(4,4,5,5-tetramethyl-1,3,2-dioxaborolan-2-yl)ethoxy)phenyl)propanoic acid (I-6.3)

To a stirred solution of methyl (2S)-2-[tert-butoxycarbonyl)amino]-3-{2-fluoro-4-[2-(4,4,5,5-tetramethyl-1,3,2-dioxaborolan-2-yl)ethoxy]phenyl}propanoate (587 mg, 1.25 mmol, 1 equiv) in H₂O (15 mL) and THF (30 mL) at room temperature was added LiOH·H₂O (105.3 mg, 2.51 mmol, 2.00 equiv) in portions. The resulting mixture was stirred at room temperature for 3 h. The mixture was acidified to “pH” 4 with 1M HCl (aq.) and extracted with EA (3×20 mL). The combined organic layers were dried over anhydrous Na₂SO₄. After filtration, the filtrate was concentrated under reduced pressure. The crude product was used in the next step directly without further purification. LCMS (ESI): mass calcd. for C₂₂H₃₃BFNO₇, 453.2; m/z found, 354 [M−Boc+H]⁺.

### Preparation of (2S)-2-[(tert-butoxycarbonyl)amino]-3-{4-[2-(dihydroxyboranyl)ethoxy]-2-fluorophenyl}propanoic Acid (I-6.4)

To a stirred solution of (2S)-2-[(tert-butoxycarbonyl)amino]-3-{2-fluoro-4-[2-(4,4,5,5-tetramethyl-1,3,2-dioxaborolan-2-yl)ethoxy]phenyl}propanoic acid (560 mg, 1.235 mmol, 1 equiv) in THF (9.0 mL) and H₂O (2.3 mL) at room temperature was added sodium periodate (792.46 mg, 3.705 mmol, 3.0 equiv) in portions. The resulting mixture was stirred at room temperature for 5 min. Then 2 N HCl (aq.) (0.49 mL, 0.988 mmol, 0.8 equiv,) was added to the above solution. The reaction was stirred at room temperature for additional 2 h. The mixture was filtered, the filter cake was washed with water (2×5 mL). The filtrate was concentrated under reduced pressure. The residue was purified by reverse flash chromatography [with the following conditions: column, C18 silica gel; mobile phase, MeCN in Water (0.1% FA), 10% to 80% gradient in 15 min; detector, UV 220 nm]. This resulted in (2S)-2-[(tert-butoxycarbonyl)amino]-3-{4-[2-(dihydroxyboranyl)ethoxy]-2-fluorophenyl}propanoic acid as a light yellow solid (280 mg, 61.08%). LCMS (ESI): mass calcd. for C₁₆H₂₃BFNO₇, 371.2; m/z found, 272.1 [M−Boc+H]⁺.

### Preparation of (2S)-2-amino-3-{4-[2-(dihydroxyboranyl)ethoxy]-2-fluorophenyl}propanoic acid (I-6)

To a stirred solution of (2S)-2-[(tert-butoxycarbonyl)amino]-3-{4-[2-(dihydroxyboranyl)ethoxy]-2-fluorophenyl}propanoic acid (280 mg, 0.755 mmol, 1 equiv) in EA (5 mL) at room temperature was added hydrogen chloride (5 mL, 20.000 mmol, 4 M in EA) dropwise. The resulting mixture was stirred at room temperature for 2 h. Then the mixture was concentrated under reduced pressure. The residue was purified by Prep-HPLC [with the following conditions (Column: YMC-Actus Triart C18, 30*150 mm, 5 μm; Mobile Phase A: Water (0.05% TFA), Mobile Phase B: ACN; Flow rate: 60 mL/min; Gradient: 10% B to 26% B in 7 min, 26% B; Wave Length: 254/220 nm; RT1(min): 5.23)] to afford (2S)-2-amino-3-{4-[2-(dihydroxyboranyl)ethoxy]-2-fluorophenyl} propanoic acid as a white solid (65 mg, 31.76%). LCMS (ESI): mass calcd. for C₁₁H₁₅BFNO₅, 271.1; m/z found, 272.1 [M+H]⁺; ¹H NMR (400 MHz, Deuterium Oxide) δ 7.13 (t, J=8.6 Hz, 1H), 6.75-6.64 (m, 2H), 4.08 (t, J=7.6 Hz, 2H), 3.86 (dd, J=7.5, 5.6 Hz, 1H), 3.18 (dd, J=14.8, 5.4 Hz, 1H), 2.97 (dd, J=14.8, 7.8 Hz, 1H), 1.24 (t, J=7.5 Hz, 2H).

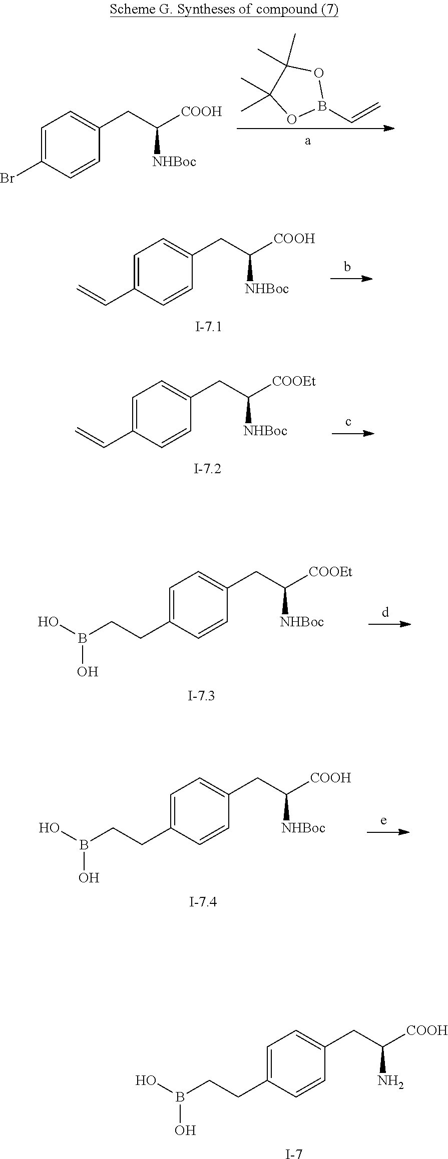

Reagents and Conditions: (a) Pd(OAc)₂, SPhos, K₃PO₄, dioxane/H₂O, 95° C.; (b) EtOH, EDCI, DMAP, DCM, rt; (c) BH₃-THF, THF, rt; (d) LiOH, H₂O, EtOH, rt; (e) TFA, DCM, rt.

### Preparation of (2S)-2-[(tert-butoxycarbonyl)amino]-3-(4-ethenylphenyl)propanoic acid (I-7.1)

To a stirred mixture of (2S)-3-(4-bromophenyl)-2-[(tert-butoxycarbonyl)amino]propanoic acid (5 g, 14.526 mmol, 1 equiv) and 2-ethenyl-4,4,5,5-tetramethyl-1,3,2-dioxaborolane (3.36 g, 21.789 mmol, 1.5 equiv) in 1,4-dioxane (100 mL) and H₂O (10 mL) at room temperature were added K3PO4 (12.33 g, 58.104 mmol, 4 equiv), SPhos (1192.71 mg, 2.905 mmol, 0.2 equiv) and Pd(OAc)2 (0.33 g, 1.453 mmol, 0.1 equiv). The resulting mixture was purged with N₂ for 1 min, and then stirred under nitrogen atmosphere at 95° C. overnight. The resulting mixture was diluted with water (50 mL), and extracted with EtOAc (3×100 mL). The resulted aqueous layer was acidified to “pH” 5 with 2N HCl (aq.), and extracted again with EtOAc (3×100 mL). The combined organic layers were washed with brine (2×100 mL), and dried over anhydrous Na₂SO₄. After filtration, the filtrate was concentrated under reduced pressure. The residue was purified by reversed-phase flash chromatography [with the following conditions: column, C18 silica gel; mobile phase, MeCN in Water (0.1% TFA), 10% to 80% gradient in 15 min; detector, UV 254/220 nm]. This resulted in (2S)-2-[(tert-butoxycarbonyl)amino]-3-(4-ethenylphenyl)propanoic acid as a brown oil (5.12 g, crude). LCMS (ESI): mass calcd. for C₁₆H₂₁NO₄, 291.1; m/z found, 290.0 [M−H]⁻.

### Preparation of ethyl (2S)-2-[(tert-butoxycarbonyl)amino]-3-(4-ethenylphenyl)propanoate (I-7.2)

To a stirred mixture of (2S)-2-[(tert-butoxycarbonyl)amino]-3-(4-ethenylphenyl)propanoic acid (5.12 g, 17.574 mmol, 1 equiv) and DMAP (0.21 g, 1.757 mmol, 0.1 equiv) in EtOH (50 mL) and DCM (100 mL) at room temperature were added EDCI (3.71 g, 19.331 mmol, 1.1 equiv) in portions. The resulting mixture was stirred at room temperature for 8h. The resulting mixture was concentrated under reduced pressure. The residue was purified by reversed-phase flash chromatography [with the following conditions: column, C18 silica gel; mobile phase, MeCN in Water (0.1% FA), 30% to 100% gradient in 15 min; detector, UV 254/220 nm]. This resulted in ethyl (2S)-2-[(tert-butoxycarbonyl)amino]-3-(4-ethenylphenyl)propanoate as a brown oil (2.86 g, 50.95%). LCMS (ESI): mass calcd. for C₁₈H₂₅NO₄, 319.1; m/z found, 220.0 [M−Boc+H]⁺.

### Preparation of 2-{4-[(2S)-2-[(tert-butoxycarbonyl)amino]-3-ethoxy-3-oxopropyl]phenyl} ethylboronic acid (I-7.3)

To a stirred mixture of ethyl (2S)-2-[(tert-butoxycarbonyl)amino]-3-(4-ethenylphenyl)propanoate (2.86 g, 8.95 mmol, 1 equiv) in THF (40 mL) under nitrogen atmosphere at 0° C. was added BH₃-THF (17.91 mL, 17.908 mmol, 2 equiv, 1M in THF) dropwise . The resulting mixture was stirred at room temperature for 2h, and then quenched by the addition of water at 0° C. The resulting mixture was stirred at room temperature for additional 2h, and then concentrated under reduced pressure. The resulting mixture was extracted with CH₂Cl₂ (3×60 mL). The combined organic layers were washed with brine (2×100 mL), and dried over anhydrous Na₂SO₄. After filtration, the filtrate was concentrated under reduced pressure. This resulted in 2-{4-[(2S)-2-[(tert-butoxycarbonyl)amino]-3-ethoxy-3-oxopropyl]phenyl}ethylboronic acid as a light brown oil (3.37 g, crude). LCMS (ESI): mass calcd. for C₁₈H₂₈BNO₆, 365.2; m/z found, 266.1 [M−Boc+H]⁺.

### Preparation of (2S)-2-[(tert-butoxycarbonyl)amino]-3-{4-[2-(dihydroxyboranyl)ethyl]phenyl}propanoic acid (I-7.4)

To a stirred mixture of 2-{4-[(2S)-2-[(tert-butoxycarbonyl)amino]-3-ethoxy-3-oxopropyl]phenyl}ethylboronic acid (3.85 g, 10.541 mmol, 1 equiv) in EtOH (60 mL) and H₂O (6 mL) at room temperature was added LiOH·H₂O (2.21 g, 52.705 mmol, 5 equiv) in portions. The resulting mixture was stirred at room temperature for 2 h, and then diluted with water (100 mL). The resulting mixture was extracted with EtOAc (3×50 mL). The resulted aqueous layer was acidified to “pH” 4-5 with 2 N HCl (aq.). The resulting mixture was extracted with EtOAc (3×100 mL). The combined organic layers were washed with brine (2×100 mL), and dried over anhydrous Na₂SO₄. After filtration, the filtrate was concentrated under reduced pressure. This resulted in (2S)-2-[(tert-butoxycarbonyl)amino]-3-{4-[2-(dihydroxyboranyl)ethyl]phenyl} propanoic acid as a light brown oil (3.37 g, 94.81%). LCMS (ESI): mass calcd. for C₁₆H₂₄BNO₆, 337.1; m/z found, 238.1 [M−Boc+H]⁺.

Preparation of (2S)-2-amino-3-{4-[2-(dihydroxyboranyl)ethyl]phenyl}propanoic acid (I-7) To a stirred mixture of (2S)-2-[(tert-butoxycarbonyl)amino]-3-{4-[2-(dihydroxyboranyl) ethyl]phenyl}propanoic acid (900 mg, 2.669 mmol, 1 equiv) in DCM (18 mL) at room temperature was added TFA (4.00 mL) dropwise. The resulting mixture was stirred at room temperature for 2 h, and then concentrated under reduced pressure. The residue was purified by Prep-HPLC [with the following conditions (Column: XBridge Shield RP18 OBD Column, 30*150 mm, 5 μm; Mobile Phase A: Water (0.1% FA), Mobile Phase B: ACN; Flow rate: 60 mL/min; Gradient: to 6% B in 6 min, 6% B; Wave Length: 254/220 nm; RT1(min): 4.77)] to afford (2S)-2-amino-3-{4-[2-(dihydroxyboranyl)ethyl]phenyl}propanoic acid as a white solid (62.5 mg, 9.88%). LCMS (ESI): mass calcd. for C₁₁H₁₆BNO₄, 237.1; m/z found, 237.9 [M+H]+; ¹H NMR (400 MHz, Methanol-d₄) δ 7.26-7.14 (m, 4H), 3.75 (dd, J=8.8, 4.2 Hz, 1H), 3.30-3.22 (m, 1H), 2.96 (dd, J=14.6, 8.9 Hz, 1H), 2.65 (t, J=8.1 Hz, 2H), 1.09 (t, J=8.1 Hz, 2H).

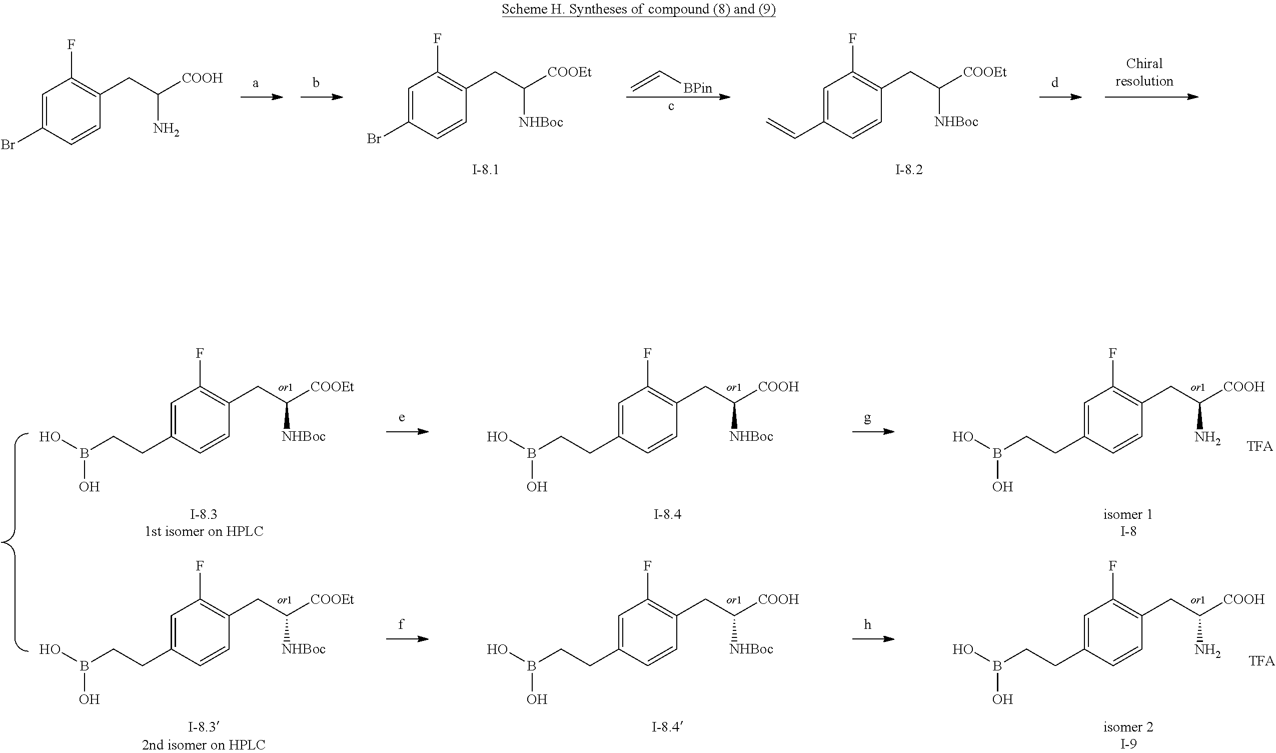

Reagents and Conditions: (a) SOCl₂, EtOH, 60° C.; (b) Boc₂O, Na₂CO₃, EtOH, rt; (c) Pd(OAc)₂, S-phos, K₃PO₄, dioxane, H₂O, 90° C.; (d) BH₃-THF, H₂O, rt, then chiral resolution; (e) LiOH, MeOH, H₂O, rt; (f) LiOH, MeOH, H₂O, rt; (g) TFA, DCM, rt; (h) TFA, DCM, rt.

### Preparation of ethyl 3-(4-bromo-2-fluorophenyl)-2-((tert-butoxycarbonyl)amino)propanoate (I-8.1)

Step A. To a stirred mixture of 2-amino-3-(4-bromo-2-fluorophenyl)propanoic acid (2 g, 7.631 mmol, 1 equiv) in ethyl alcohol (30 mL) was added thionyl chloride (4.54 g, 38.155 mmol, 5 equiv) dropwise at room temperature. The mixture was stirred at 60° C. for 8 h, and then concentrated under reduced pressure. The residue was basified to “pH” 8 with saturated Na₂CO₃ (aq.), and then extracted with DCM (3×100 mL). The combined organic layers were washed with brine (2×100 mL), dried over anhydrous Na₂SO₄. After filtration, the filtrate was concentrated under reduced pressure to afford ethyl 2-amino-3-(4-bromo-2-fluorophenyl)propanoate as a light yellow solid (2.5 g, crude), which was used directly in the next step.

Step B. To a stirred mixture of ethyl 2-amino-3-(4-bromo-2-fluorophenyl)propanoate (2.1 g, 7.24 mmol, 1 equiv) and Na₂CO₃ (3.07 g, 28.95 mmol, 4 equiv) in EtOH (50 mL) at room temperature was added di-tert-butyl dicarbonate (3.16 g, 14.48 mmol, 2 equiv). The reaction mixture was stirred at 25° C. for 8 h, and then concentrated under reduced pressure. The residue was diluted with water, and extracted with DCM (3×100 mL). The combined organic layers were washed with brine (2×200 mL), dried over anhydrous Na₂SO₄. After filtration, the filtrate was concentrated under reduced pressure to afford ethyl 3-(4-bromo-2-fluorophenyl)-2-[(tert-butoxycarbonyl) amino]propanoate as a light yellow solid (4.6 g, crude). LCMS (ESI): mass calcd. for C₁₆H₂₁BrFNO₄, 389.1; m/z found, 290.0 [M−Boc+H]⁺.

Preparation of ethyl 2-((tert-butoxycarbonyl)amino)-3-(2-fluoro-4-vinylphenyl)propanoate (I-8.2)

To a stirred mixture of ethyl 3-(4-bromo-2-fluorophenyl)-2-[(tert-butoxycarbonyl) amino]propanoate (4.2 g, 10.762 mmol, 1 equiv) and 2-ethenyl-4,4,5,5-tetramethyl-1,3,2-dioxaborolane (2.49 g, 16.143 mmol, 1.5 equiv) in dioxane (80 mL) and H₂O (8 mL) at room temperature was added K₃PO₄ (9.14 g, 43.048 mmol, 4 equiv), S-Phos (0.88 g, 2.152 mmol, 0.2 equiv) and Pd(OAc)2 (0.24 g, 1.076 mmol, 0.1 equiv). The reaction mixture was purged with N₂ for 1 min, and then stirred under nitrogen atmosphere at 90° C. for 8 h. The resulting mixture was concentrated under reduced pressure. The residue was purified by silica gel column chromatography, eluted with PE/EA (5:1) to afford ethyl 2-[(tert-butoxycarbonyl)amino]-3-(4-ethenyl-2-fluorophenyl)propanoate as a light yellow solid (2.1 g, 57.83%). LCMS (ESI): mass calcd. for C₁₈H₂₄FNO₄, 337.2; m/z found, 238.2 [M−Boc+H]⁺.

### Preparation of 2-{4-[(25)-2-[(tert-butoxycarbonyl)amino]-3-ethoxy-3-oxopropyl]-3-fluorophenyl}ethylboronic acid (1-8.3)and 2-{4-[(2R)-2-[(tert-butoxycarbonyl) amino]-3-ethoxy-3-oxopropyl]-3-fluorophenyl}ethylboronic acid (I-8.3′)

To a stirred mixture of ethyl 2-[(tert-butoxycarbonyl)amino]-3-(4-ethenyl-2-fluorophenyl) propanoate (2 g, 5.928 mmol, 1 equiv) in THF (40 mL) at 0° C. was added borane solution (1M in THF, 5.9 mL, 11.86 mmol, 2 equiv) dropwise. The mixture was stirred at 25° C. for 2 h, and then quenched by the addition of water at 0° C. The mixture was stirred at 25° C. for additional 2 h, and then concentrated under reduced pressure. The residue was purified by Prep-HPLC [with the following conditions (Column: Sunfire prep C18 column, 30*150 mm, 5)..tm; Mobile Phase A: Water (0.05% TFA), Mobile Phase B: MeOH—Preparative; Flow rate: 60 mL/min; Gradient: 55% B to 70% B in 11 min, 70% B; Wave Length: 254/220 nm; RT1(min): 9.48)] to afford 2-(4-{2-[(tert-butoxycarbonyl)amino]-3-ethoxy-3-oxopropyl}-3-fluorophenyl)ethyl-boronic acid (560 mg, 24.65%) as a colorless oil.

Then, the product (560 mg) was separated by Prep-SFC with the following conditions (Column: CHIRALCEL AY-H, 2*25 cm, 5 μm; Mobile Phase A: CO₂, Mobile Phase B: EtOH-HPLC; Flow rate: 40 mL/min; Gradient: isocratic 10% B; Column Temperature (° C.): 35; Back Pressure(bar): 100; Wave Length: 220 nm; RT1 (min): 2.87; RT2 (min): 3.77; Sample Solvent: MeOH—Preparative; Injection Volume: 0.2 mL) to afford 2-14-[(2S*)-2-[(tert-butoxycarbonyl)amino]-3-ethoxy-3-oxopropyl]-3- fluorophenyljethylboronic acid (I-8.3, 132 mg, 23.57%, ee>99%, 1^{st} isomer on HPLC) as an off-white solid and 2-14-[(2R*)-2-[(tert-butoxycarbonyl)amino]-3-ethoxy-3-oxopropyl]- 3-fluorophenyljethylboronic acid (I-8.3′,135 mg, 24.11%, ee>97%, 2^{nd} isomer on HPLC) as an off-white solid. LCMS (ESI): mass calcd. for C₁₈H₂₇BFNO₆, 383.2; m/z found, 284.0 [M−Boc+H]⁺.

### Preparation of (S*)-3-(4-(2-boronoethyl)-2-fluorophenyl)-2-((tert-butoxycarbonyl)amino) propanoic acid (I-8.4)

To a stirred mixture of 2-{4-[(2S*)-2-[(tert-butoxycarbonyl)amino]-3-ethoxy-3-oxopropyl]-3-fluorophenyl}ethylboronic acid (130 mg, 0.339 mmol, 1 equiv) in MeOH (2 mL) and H₂O (2 mL) at room temperature was added LiOH·H₂O (71.12 mg, 1.695 mmol, 5 equiv). The mixture was stirred at 25° C. for 2 h, and then acidified to “pH” 4 with 2N HCl (aq.). The resulting mixture was concentrated under reduced pressure. The residue was purified by reversed-phase flash chromatography [with the following conditions: column, C18 silica gel; mobile phase, ACN in Water (0.1% FA), 10% to 60% gradient in 10 min; detector, UV 254/220 nm] to afford (2S *)-2-[(tert-butoxycarbonyl)amino]-3-{4-[2-(dihydroxyboranyl)ethyl]-2-fluorophenyl}propanoic acid (100 mg, crude) as an off-white solid. LCMS (ESI): mass calcd. for C₁₆H₂₃BFNO₆, 355.2; m/z found, 255.95 [M−Boc+H]⁺.

Preparation of (R*)-3-(4-(2-boronoethyl)-2-fluorophenyl)-2-((tert-butoxycarbonyl)amino) propanoic acid (I-8.4′)

To a stirred mixture of 2-{4-[(2R*)-2-[(tert-butoxycarbonyl)amino]-3-ethoxy-3-oxopropyl]-3-fluorophenyl}ethylboronic acid (130 mg, 0.339 mmol, 1 equiv) in MeOH (2 mL) and H₂O (2 mL) at room temperature was added LiOH·H₂O (71.12 mg, 1.695 mmol, 5 equiv). The mixture was stirred at 25° C. for 2 h, and then acidified to “pH” 4 with 2N HCl (aq.). The resulting mixture was concentrated under reduced pressure. The residue was purified by reversed-phase flash chromatography [with the following conditions: column, C18 silica gel; mobile phase, MeCN in Water (0.1% FA), 10% to 60% gradient in 10 min; detector, UV 254/220 nm] to afford (2R*)-2-[(tert-butoxycarbonyl)amino]-3-{4-[2-(dihydroxyboranyl)ethyl]-2-fluorophenyl}propanoic acid as an off-white solid(100 mg, crude). LCMS (ESI): mass calcd. for C₁₆H₂₃BFNO₆, 355.2; m/z found, 256.0 [M−Boc+H]⁺.

### Preparation of (S*)-2-amino-3-(4-(2-boronoethyl)-2-fluorophenyl)propanoic acid (I-8)

To a stirred mixture of (2S*)-2-[(tert-butoxycarbonyl)amino]-3-{4-[2-(dihydroxyboranyl)ethyl]-2-fluorophenyl}propanoic acid (100 mg, 0.282 mmol, 1 equiv) in DCM (3 mL) was added TFA(0.5 mL) dropwise at room temperature. The mixture was stirred at 25° C. for 2 h, and then concentrated under reduced pressure. The residue was purified by Prep-HPLC [with the following conditions (Column: XBridge Shield RP18 OBD Column, 19*150 mm, 5 μm; Mobile Phase A: Water (0.1% FA), Mobile Phase B: MeOH-HPLC; Flow rate: 25 mL/min; Gradient: 5% B to 17% B in 6 min, 17% B; Wave Length: 220 nm; RT1(min): 4.72)] to afford (2S*)-2-amino-3-{4-[2-(dihydroxyboranyl)ethyl]-2-fluorophenyl} propanoic acid; trifluoroacetic acid (56.9 mg, 54.76%) as an off-white solid. LCMS (ESI): mass calcd. for C₁₁H₁₅BFNO₄, 255.2; m/z found, 255.9 [M+H]⁺; ¹H NMR (400 MHz, Methanol-d₄) δ 7.22 (t, J=7.8 Hz, 1H), 7.07-6.87 (m, 2H), 4.07-3.87 (m, 1H), 3.41-3.32 (m, 1H), 3.05 (dd, J=14.6, 7.9 Hz, 1H), 2.67 (t, J=8.1 Hz, 2H), 1.10 (t, J=8.2 Hz, 2H).

### Preparation of (R*)-2-amino-3-(4-(2-boronoethyl)-2-fluorophenyl)propanoic acid (I-9)

To a stirred mixture of (2R*)-2-[(tert-butoxycarbonyl)amino]-3-{4-[2-(dihydroxyboranyl)ethyl]- 2-fluorophenyl}propanoic acid (110 mg, 0.310 mmol, 1 equiv) in DCM (3 mL) was added TFA (0.5 mL) dropwise at room temperature. The mixture was stirred at 25° C. for 2 h, and then concentrated under reduced pressure. The residue was purified by Prep-HPLC [with the following conditions (Column: XBridge Shield RP18 OBD Column, 19*150 mm, 5 μm; Mobile Phase A: Water (0.1% FA), Mobile Phase B: MeOH-HPLC; Flow rate: 25 mL/min; Gradient: 5% B to 20% B in 7 min, 20% B; Wave Length: 220 nm; RT1 (min): 4.85)] to afford (2R*)-2-amino-3-{4-[2-(dihydroxyboranyl)ethyl]-2-fluorophenyl} propanoic acid; trifluoroacetic acid as an off-white solid (38.3 mg, 33.18%). LCMS (ESI): mass calcd. for C₁₁H₁₅BFNO₄, 255.2; m/z found, 256.15 [M+H]⁺; ¹H NMR (400 MHz, Methanol-d₄) δ 7.23 (t, J=7.8 Hz, 1H), 7.06-6.87 (m, 2H), 3.91-3.71 (m, 1H), 3.41-3.32 (m, 1H), 2.99 (dd, J=14.5, 8.5 Hz, 1H), 2.67 (t, J=8.0 Hz, 2H), 1.10 (t, J=8.0 Hz, 2H).

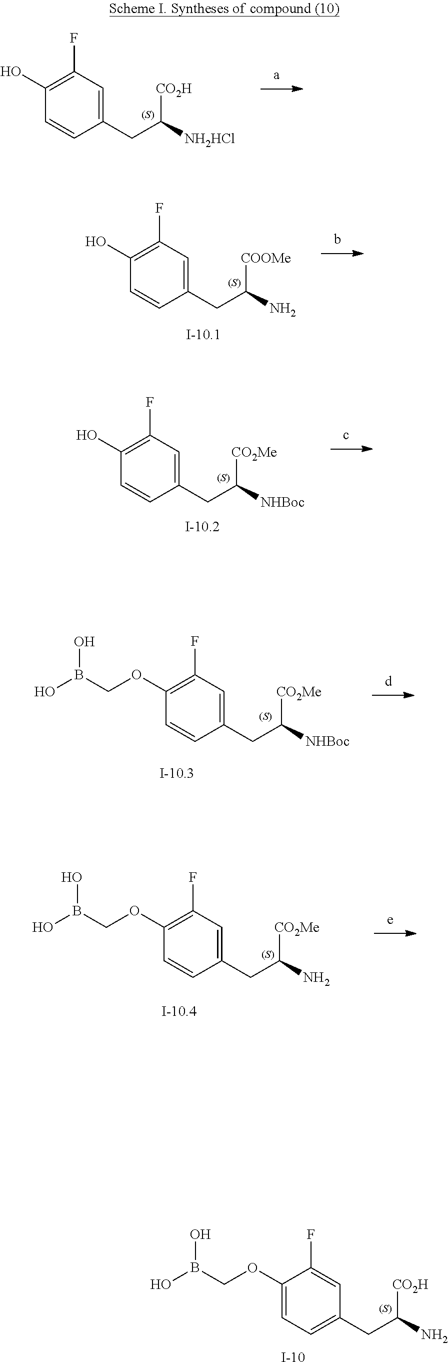

Reagents and Conditions: (a) SOCl₂, MeOH, 50° C.; (b) Boc₂O, TEA, DCM, THF; (c) K₂CO₃, DMF, 2-(iodomethyl)-4,4,5,5-tetramethyl-1,3,2-dioxaborolane; (d) HCl in EA, EA; (e) LiOH H₂O, THF, H₂O.

### Preparation of methyl (2S)-2-amino-3-(3-fluoro-4-hydroxyphenyl)propanoate (I-10.1)

To a stirred mixture of 3-fluorotyrosine (1 g, 5.021 mmol, 1 equiv) in methanol (20 mL) under nitrogen atmosphere at 0° C. was added thionyl chloride (3.58 g, 30.126 mmol, 6 equiv) dropwise. The resulting mixture was stirred under nitrogen atmosphere at 50° C. for 4 h, and then concentrated. The residue was basified to “pH” 8 with saturated NaHCO₃ (aq.). The residue was purified by reverse flash chromatography [with the following conditions: column, C18 silica gel; mobile phase, MeCN in Water (0.1% NH₃·H₂O), 30% to 40% gradient in 8 min; detector, UV 254/220 nm]. This resulted in methyl (2S)-2-amino-3-(3-fluoro-4-hydroxyphenyl)propanoate as a white solid (920 mg, 85.95%). LCMS (ESI): mass calcd. for Cioth2FN03, 213.1; m/z found, 214.3 [M+H]⁺.

### Preparation of methyl (2S)-2-[(tert-butoxycarbonyl)Amino]-3-(3-fluoro-4-hydroxyphenyl) propanoate (I-10.2)

To a stirred mixture of methyl (2S)-2-amino-3-(3-fluoro-4-hydroxyphenyl)propanoate (860 mg, 4.034 mmol, 1 equiv) and TEA (612.26 mg, 6.051 mmol, 1.5 equiv) in DCM/THF (10 mL/10 mL) under nitrogen atmosphere at room temperature was added di-tert-butyl dicarbonate (1056.40 mg, 4.841 mmol, 1.2 equiv) . The resulting mixture was stirred under nitrogen atmosphere at room temperature overnight, and then concentrated under reduced pressure. The residue was purified by reverse flash chromatography [with the following conditions: column, C18 silica gel; mobile phase, MeCN in Water (0.1% NH₃·H₂O), 30% to 50% gradient in 8 min; detector, UV 254/220 nm]. This resulted in methyl (2S)-2-Rtert-butoxycarbonyl)aminol-3-(3-fluoro-4-hydroxyphenyl)propanoate as a white solid (924 mg, 73.11%). LCMS (ESI): mass calcd. for C₁₅H₂₀FNO₅, 313.1; m/z found, 314.2 [M+H]⁺.

### Preparation of 4-[(2S)-2-[(tert-butoxycarbonyl)Amino]-3-methoxy-3-oxopropyl]-2-fluorophenoxyrnethylboronic acid (I-10.3)

To a stirred mixture of methyl (2S)-2-Rtert-butoxycarbonyl)aminol-3-(3-fluoro-4-hydroxyphenyl)propanoate (924 mg, 2.949 mmol, 1 equiv) and K₂CO₃ (2.04 g, 14.745 mmol, 5 equiv) in DMF (18 mL) under nitrogen atmosphere at room temperature was added 2-(iodomethyl)-4,4,5,5-tetramethyl-1,3,2-dioxaborolane (3.95 g, 14.744 mmol, 5.00 equiv) . The resulting mixture was stirred under nitrogen atmosphere at room temperature overnight. The reaction mixture was purified directly by reverse flash chromatography [with the following conditions: column, C18 silica gel; mobile phase, MeCN in Water (0.1% FA), 30% to 50% gradient in 8 min; detector, UV 254/220 nm]. This resulted in 4-[(2S)-2-[(tert-butoxycarbonyl)amino]-3-methoxy-3-oxopropyl]-2- fluorophenoxymethylboronic acid as a white solid (630 mg, 57.56%). LCMS (ESI): mass calcd. for C₁₆H₂₃FNO₇, 371.2; m/z found, 272.1 [M+H−Boc]⁺.

### Preparation of 4-[(2S)-2-amino-3-methoxy-3-oxopropyl]-2-fluorophenoxymethylboronic acid (I-10.4)

To a stirred mixture of 4-[(2S)-2-[(tert-butoxycarbonyl)amino]-3-methoxy-3-oxopropyl]-2-fluorophenoxymethylboronic acid (900 mg, 2.43 mmol, 1 equiv) in EtOAc (10 mL) under nitrogen atmosphere at room temperature was added hydrogen chloride (5 mL, 4 M in EtOAc) dropwise . The resulting mixture was stirred under nitrogen atmosphere at room temperature for 3 h, and then concentrated under reduced pressure. The residue was purified by reverse flash chromatography [with the following conditions: column, C18 silica gel; mobile phase, MeCN in Water (0.1% FA), 15% to 40% gradient in 7 min; detector, UV 254/220 nm]. This resulted in 4-[(2S)-2-amino-3-methoxy-3-oxopropyl]-2-fluorophenoxymethylboronic acid (550 mg, 83.68%) as a white solid. LCMS (ESI): mass calcd. for C₁₁H₁₅BFNO₅, 271.1; m/z found, 272.3 [M+H]⁺.

### Preparation of (2S)-2-amino-3-{4-[(dihydroxyboranyl)methoxy]-3-fluorophenyl}propanoic acid (I-10)

To a stirred mixture of 4-[(2S)-2-amino-3-methoxy-3-oxopropyl]-2-fluorophenoxymethylboronic acid (550 mg, 2.029 mmol, 1 equiv) in THF/H₂O (8 mL/8 mL) under nitrogen atmosphere at room temperature was added LiOH·H₂O (255.43 mg, 6.087 mmol, 3 equiv) . The resulting mixture was stirred under nitrogen atmosphere at room temperature for 4 h, and then acidified to “pH” 6 with 2N HCl (aq.). The resulting mixture was concentrated under vacuum. The residue was purified by reverse flash chromatography [with the following conditions: column, C18 silica gel; mobile phase, MeCN in Water (0.1% FA), 0% to 5% gradient in 8 min; detector, UV 254/220 nm]. The product was further purified by Prep-HPLC [with the following conditions (Column: Atlantis Prep T3 OBD Column, 19*150mm 5 μm; Mobile Phase A: Water (0.1% TFA), Mobile Phase B: ACN; Flow rate: 25 mL/min; Gradient: 8% B to 22% B in 7 min, 22% B; Wave Length: 254/220 nm; RT1(min): 5.04)]. This resulted in (2S)-2-amino-3-{4-[(dihydroxyboranyl)methoxy]-3-fluorophenyl} propanoic acid; trifluoroacetic acid as a white solid (68 mg, 13.04%). LCMS (ESI): mass calcd. for C₁₀H₁₃BFNO₅, 257.1; m/z found, 258.1 [M+H]⁺. ¹H NMR (400 MHz, Deuterium Oxide) δ 7.12-6.96 (m, 3H), 3.95 (dd, J=7.7, 5.3 Hz, 1H), 3.85 (s, 2H), 3.17 (dd, J=14.7, 5.3 Hz, 1H), 3.03 (dd, J=14.7, 7.7 Hz, 1H).

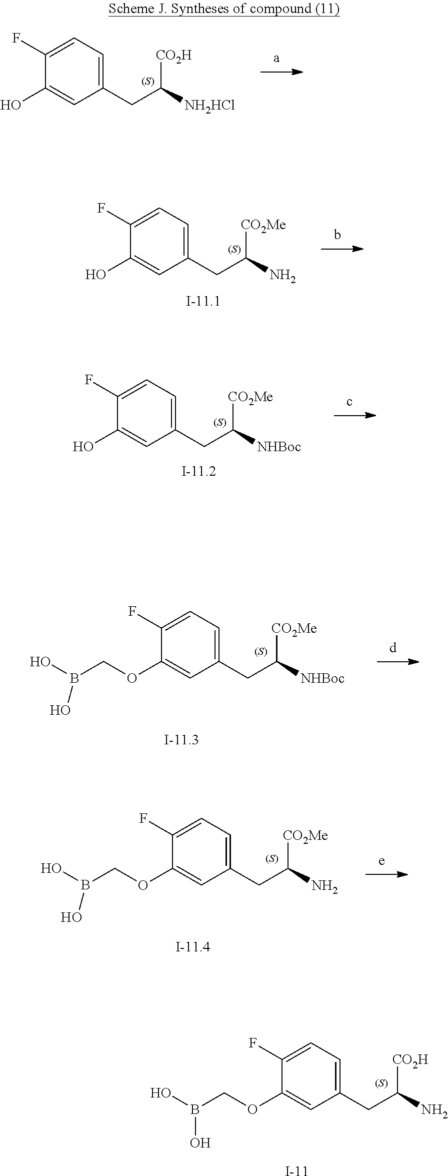

Reagents and Conditions: (a) SOCl₂, MeOH, 50° C.; (b) Boc₂O, DCM, TEA; (c) K₂CO₃, DMF, 2-(iodomethyl)-4,4,5,5-tetramethyl-1,3,2-dioxaborolane; (d) HCl in EA, EA; (e) LiOH·H₂O, THF, H₂O.

### Preparation of methyl (2S)-2-amino-3-(4-fluoro-3-hydroxyphenyl)propanoate (I-11.1)

To a stirred mixture of (2S)-2-amino-3-(4-fluoro-3-hydroxyphenyl)propanoic acid hydrochloride (900 mg, 3.819 mmol, 1 equiv) in methanol (18 mL) under nitrogen atmosphere at 0° C. was added thionyl chloride (5 mL) . The resulting mixture was stirred under nitrogen atmosphere at 50° C. overnight, and then concentrated under vacuum. The residue was basified to “pH” 8 with saturated Na₂CO₃ (aq.). The mixture was purified by reverse flash chromatography [with the following conditions: column, C18 silica gel; mobile phase, MeCN in Water (10 mmol/L NH4HCO3), 30% to 40% gradient in 10 min; detector, UV 254/220 nm]. This resulted in methyl (2S)-2-amino-3-(4-fluoro-3-hydroxyphenyl)propanoate as a white solid (680 mg, 83.50%). LCMS (ESI): mass calcd. for C₁₀H₁₂FNO₃, 213.1; m/z found, 214.1 [M+H]⁺.

### Preparation of methyl (2S)-2-[(tert-butoxycarbonyl)amino]-3-(4-fluoro-3-hydroxyphenyl)propanoate (I-11.2)

To a stirred mixture of methyl (2S)-2-amino-3-(4-fluoro-3-hydroxyphenyl)propanoate (800 mg, 3.752 mmol, 1 equiv) and TEA (759.39 mg, 7.504 mmol, 2 equiv) in DCM (16 mL) under nitrogen atmosphere at room temperature was added di-tert-butyl dicarbonate (982.70 mg, 4.502 mmol, 1.2 equiv) . The resulting mixture was stirred under nitrogen atmosphere at room temperature overnight, and then concentrated under reduced pressure. The residue was purified by reverse flash chromatography [with the following conditions: column, C18 silica gel; mobile phase, MeCN in Water (0.1% NH₃·H₂O), 50% to 60% gradient in 7 min; detector, UV 254/220 nm]. This resulted in methyl (2S)-2-[(tert-butoxycarbonyl)amino]-3-(4-fluoro-3-hydroxyphenyl)propanoate as a white solid (297 mg, 25.26%). LCMS (ESI): mass calcd. for C₁₅H₂₀FN05, 313.1; m/z found, 312.2 [M−H]⁻.

### Preparation of 5-[(2S)-2-[(tert-butoxycarbonyl)amino]-3-methoxy-3-oxopropyl]-2-fluoro-phenoxyrnethylboronic acid (I-11.3)

To a stirred mixture of methyl (2S)-2-[(tert-butoxycarbonyl)amino]-3-(4-fluoro-3-hydroxyphenyl)propanoate (488 mg, 1.557 mmol, 1 equiv) and K₂CO₃ (1.08 g, 7.785 mmol, 5 equiv) in DMF (9 mL) under nitrogen atmosphere at room temperature was added 2-(iodomethyl)-4,4,5,5-tetramethyl-1,3,2-dioxaborolane (2.09 g, 7.785 mmol, 5 equiv) . The resulting mixture was stirred under nitrogen atmosphere at room temperature overnight. The reaction mixture was purified directly by reverse flash chromatography [with the following conditions: column, C18 silica gel; mobile phase, MeCN in Water (0.1% FA), 30% to 55% gradient in 8 min; detector, UV 254/220 nm]. This resulted in 5-[(2S)-2-[(tert-butoxycarbonyl)amino]-3-methoxy-3-oxopropyl]-2- fluorophenoxymethylboronic acid as a white solid (434 mg, 75.07%). LCMS (ESI): mass calcd. for C₁₆H₂₃BFNO₇, 371.2; m/z found, 272.3 [M+H−Boc]⁺.

### Preparation of 5-[(2S)-2-amino-3-methoxy-3-oxopropyl]-2-fluorophenoxymethylboronic acid (I-11.4)

To a stirred mixture of 5-[(2S)-2-[(tert-butoxycarbonyl)amino]-3-methoxy-3-oxopropyl]-2-fluorophenoxymethylboronic acid (290 mg, 0.781 mmol, 1 equiv) in EtOAc (3 mL) under nitrogen atmosphere at room temperature was added HCl (1 mL, 4 N in EtOAc) dropwise . The resulting mixture was stirred under nitrogen atmosphere at room temperature overnight. The resulting mixture was concentrated under vacuum. The residue was purified by reverse flash chromatography [with the following conditions: column, C18 silica gel; mobile phase, MeCN in Water (0.1% FA), 0% to 5% gradient in 10 min; detector, UV 254/220 nm]. This resulted in 5-[(2S)-2-amino-3-methoxy-3-oxopropyl]-2-fluorophenoxymethylboronic acid as a white solid (167 mg, 78.86%). LCMS (ESI): mass calcd. for C₁₁H₁₅BFNO₃, 271.1; m/z found, 272.2 [M+H]⁺.

### Preparation of (2S)-2-amino-3-{3-[(dihydroxyboranyl)methoxy]-4-fluorophenyl}propanoic acid (I-11)

To a stirred mixture of 5-[(2S)-2-amino-3-methoxy-3-oxopropyl]-2-fluorophenoxymethylboronic acid (317 mg, 1.170 mmol, 1 equiv) in THF/H₂O (6 mL/3 mL) under nitrogen atmosphere at room temperature was added LiOH·H₂O (147.22 mg, 3.510 mmol, 3 equiv). The resulting mixture was stirred under nitrogen atmosphere at room temperature for 3 h, and then acidified to “pH” 6 with 2N HCl (aq.). The resulting mixture was concentrated under vacuum. The residue was purified by reverse flash chromatography [with the following conditions: column, C18 silica gel; mobile phase, MeCN in Water (0.1% FA), 0% to 5% gradient in 8 min; detector, UV 254/220 nm. The product was further purified by Prep-HPLC with the following conditions (Column: YMC-Actus Triart C18, 30*150 mm, 5 μm; Mobile Phase A: Water (0.1% FA), Mobile Phase B: ACN; Flow rate: 60 mL/min; Gradient: 5% B to 20% B in 7 min, 20% B; Wave Length: 254/220 nm; RT1(min): 3.58)]. This resulted in (2S)-2-amino-3-{3-[(dihydroxyboranyl)methoxy]-4-fluorophenyl}propanoic acid as a white solid (60 mg, 19.04%). LCMS (ESI): mass calcd. for C₁₀H₁₃BFNO₅, 257.1; m/z found, 258.2 [M+H]⁺. ¹H NMR (400 MHz, Deuterium Oxide) 6 7.07 (dd, J=11.5, 8.3 Hz, 1H), 6.98 (d, J=8.3 Hz, 1H), 6.82-6.75 (m, 1H), 3.93-3.87 (m, 1H), 3.82 (s, 2H), 3.17 (dd, J=14.6, 5.2 Hz, 1H), 3.02 (dd, J=14.6, 7.9 Hz, 1H).

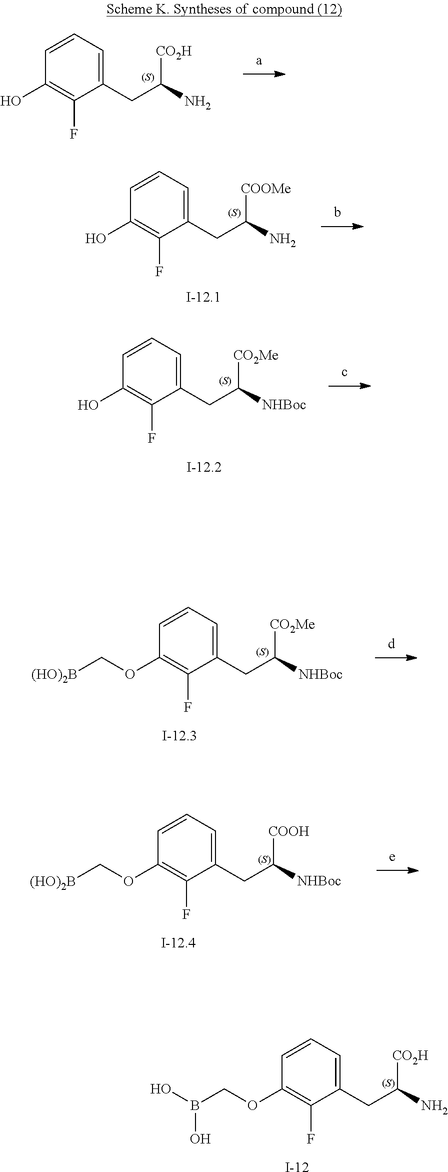

Reagents and Conditions: (a) SOCl₂, MeOH, 50° C.; (b) Boc₂O, NaHCO₃, dioxane, H₂O; (c) K₂CO₃, DMF, 2-(iodomethyl)-4,4,5,5-tetramethyl-1,3,2-dioxaborolane; (d) LiOH H₂O, THF, H₂O; (e) HCl in EA, EA.

### Preparation of methyl (2S)-2-amino-3-(2-fluoro-3-hydroxyphenyl)propanoate (I-12.1)

To a stirred mixture of (2S)-2-amino-3-(2-fluoro-3-hydroxyphenyl)propanoic acid (800 mg, 4.016 mmol, 1 equiv) in methanol (16 mL) under nitrogen atmosphere at 0° C. was added thionyl chloride (8 mL) dropwise . The resulting mixture was stirred under nitrogen atmosphere at 50° C. for 4 h. The resulting mixture was concentrated. The residue was basified to “pH” 8 with saturated NaHCO₃ (aq.). The residue was purified by reverse flash chromatography [with the following conditions: column, C18 silica gel; mobile phase, MeCN in Water (0.1% NH₃·H₂O), 10% to 20% gradient in 8 min; detector, UV 254/220 nm]. This resulted in methyl (2S)-2-amino-3-(2-fluoro-3-hydroxyphenyl)propanoate as a white solid (790 mg, 92.25%). LCMS (ESI): mass calcd. for C₁₀H₁₂FNO₃, 213.1; m/z found, 214.3 [M+H]⁺.

### Preparation of methyl (2S)-2-[(tert-butoxycarbonyl)amino]-3-(2-fluoro-3-hydroxyphenyl) propanoate (I-12.2)

To a stirred mixture of methyl (2S)-2-amino-3-(2-fluoro-3-hydroxyphenyl)propanoate (2.72 g, 12.8 mmol, 1 equiv) and NaHCO₃ (2.68 g, 31.9 mmol, 2.5 equiv) in dioxane/H₂O (27 mL/27 mL) under nitrogen atmosphere at room temperature was added di-tert-butyl dicarbonate (4.18 g, 19.1 mmol, 1.5 equiv) in portions . The resulting mixture was stirred under nitrogen atmosphere at room temperature for 5 h. The resulting mixture was filtered, and the filtering pad was washed with MeOH (3×10 mL). The filtrate was concentrated under reduced pressure. The residue was purified by reverse flash chromatography [with the following conditions: column, C18 silica gel; mobile phase, MeCN in Water (0.1% NH₃·H₂O), 30% to 50% gradient in 8 min; detector, UV 254/220 nm]. This resulted in methyl (2S)-2-[(tert-butoxycarbonyl)amino]-3-(2-fluoro-3-hydroxyphenyl)propanoate as a white solid (1.914 g, 47.88%). LCMS (ESI): mass calcd. for C₁₅H₂₀FN05, 313.1; m/z found, 312.2 [M−H]⁻.

### Preparation of 3-[(2S)-2-[(tert-butoxycarbonyl)amino]-3-methoxy-3-oxopropyl]-2-fluorophenoxymethylboronic acid (1-12.3)

To a stirred mixture of methyl (2S)-2-[(tert-butoxycarbonyl)amino]-3-(2-fluoro-3-hydroxyphenyl)propanoate (286 mg, 0.913 mmol, 1 equiv) and K₂CO₃ (630.76 mg, 4.565 mmol, 5 equiv) in DMF (5 mL) under nitrogen atmosphere at room temperature was added 2-(iodomethyl)-4,4,5,5-tetramethyl-1,3,2-dioxaborolane (1.2 g, 4.565 mmol, 5 equiv) . The resulting mixture was stirred under nitrogen atmosphere at room temperature overnight. The reaction mixture was purified directly by reverse flash chromatography [with the following conditions: column, C18 silica gel; mobile phase, MeCN in Water (0.1% FA), 30% to 45% gradient in 8 min; detector, UV 254/220 nm]. This resulted in 3-[(2S)-2-[(tert-butoxycarbonyl)amino]-3-methoxy-3-oxopropyl]-2- fluorophenoxymethylboronic acid as a white solid (210 mg, 61.98%). LCMS (ESI): mass calcd. for C₁₆H₂₃BFNO₇, 371.2; m/z found, 272.1 [M+H−Boc]⁺.

### Preparation of (2S)-2-[(tert-butoxycarbonyl)amino]-3-{3-[(dihydroxyboranyl)methoxy]-2-fluorophenyl}propanoic acid (I-12.4)

To a stirred mixture of 3-[(2S)-2-[(tert-butoxycarbonyl)amino]-3-methoxy-3-oxopropyl]-2-fluorophenoxymethylboronic acid (302 mg, 0.814 mmol, 1 equiv) in THF/H₂O (6 mL/3 mL) was under nitrogen atmosphere at room temperature added LiOH·H₂O (58.46 mg, 2.442 mmol, 3 equiv) . The resulting mixture was stirred under nitrogen atmosphere at room temperature for 3 h, and then acidified to “pH” 6 with 1N HCl (aq.). The resulting mixture was concentrated under vacuum. The residue was purified by reverse flash chromatography [with the following conditions: column, C18 silica gel; mobile phase, MeCN in Water (0.1% FA), 20% to 35% gradient in 8 min; detector, UV 254/220 nm]. This resulted in (2S)-2-[(tert-butoxycarbonyl)amino]-3-{3-[(dihydroxyboranyl)methoxy]-2-fluorophenyl}propanoic acid as a white solid (210 mg, 72.27%). LCMS (ESI): mass calcd. for C₁₅H₂₁BFNO₇, 357.1; m/z found, 356.3 [M−H]⁻.

### Preparation of (2S)-2-amino-3-{3-[(dihydroxyboranyl)methoxy]-2-fluorophenyl}propanoic acid; trifluoroacetic acid (I-12)

To a stirred mixture of (2S)-2-[(tert-butoxycarbonyl)amino]-3-{3-[(dihydroxyboranyl)methoxy]-2-fluorophenyl}propanoic acid (210 mg, 0.588 mmol, 1 equiv) in EtOAc (2 mL) under nitrogen atmosphere at room temperature was added HCl (2 mL, 4 M in EtOAc) . The resulting mixture was stirred under nitrogen atmosphere at room temperature for 2 h, and then concentrated under vacuum. The residue was purified by Prep-HPLC [with the following conditions (Column: XBridge Prep Amide OBD Column, 19*150 mm, 5 μm; Mobile Phase A: Water (0.05% TFA), Mobile Phase B: ACN; Flow rate: 25 mL/min; Gradient: 95% B to 83% B in 2 min, 83% B to 60% B in 10 min, 60% B; Wave Length: 220 nm; RT1(min): 7.79)]. This resulted in (2S)-2-amino-3-{3-[(dihydroxyboranyl)methoxy]-2-fluorophenyl}propanoic acid; trifluoroacetic acid as a white solid (55 mg, 36.39%). LCMS (ESI): mass calcd. for C₁₀H₁₃BFNO₅, 257.1; m/z found, 258.1 [M+H]⁺. ¹H NMR (400 MHz, Deuterium Oxide) δ 7.10-6.98 (m, 2H), 6.85-6.75 (m, 1H), 4.06 (dd, J=7.6, 5.8 Hz, 1H), 3.81 (s, 2H), 3.29 (dd, J=14.5, 5.7 Hz, 1H), 3.10 (dd, J=14.7, 7.7 Hz, 1H).

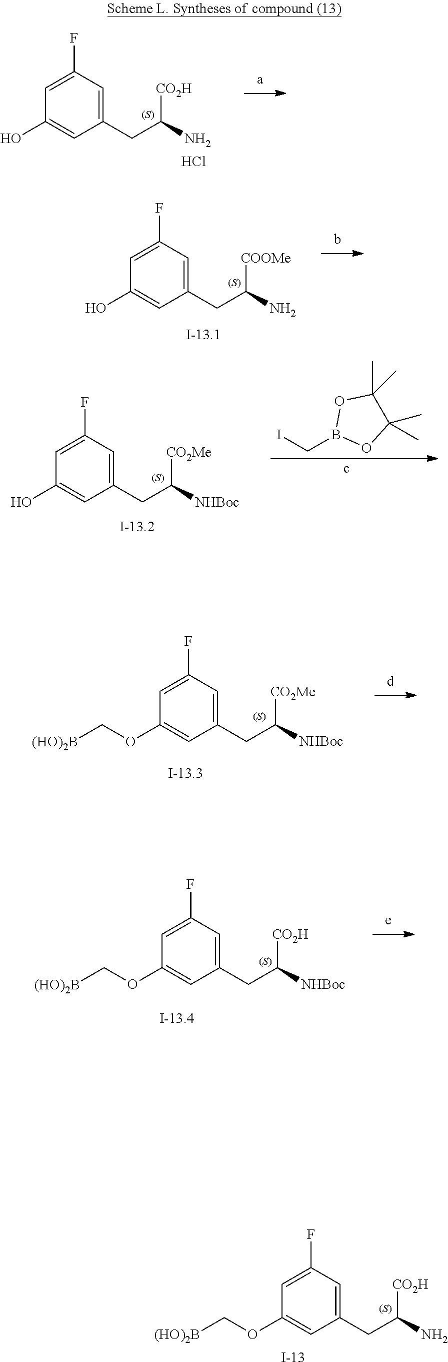

Reagents and Conditions: (a) SOCl₂, MeOH, 50° C.; (b) Boc₂O, NaHCO₃, dioxane, H₂O, rt; (c) 2-(bromomethyl)-4,4,5,5-tetramethyl-1,3,2-dioxaborolane, K₂CO₃, DMF, rt; (d) LiOH·H₂O, THF, H₂O, rt; (e) HCl in EtOAc (4 M), EtOAc, rt.

### Preparation of methyl (S)-2-amino-3-(3-fluoro-5-hydroxyphenyl)propanoate (1-13.1)

To a stirred mixture of (2S)-2-amino-3-(3-fluoro-5-hydroxyphenyl)propanoic acid (1 g, 5.02 mmol, 1 equiv) in MeOH (20 mL) under nitrogen atmosphere at 0° C. was added SOCl₂ (10 mL) dropwise . The resulting mixture was stirred under nitrogen atmosphere at 50° C. overnight, and then concentrated under vacuum. The residue was basified to “pH” 8 with saturated NaHCO₃ (aq.). The resulting mixture was extracted with EtOAc (3×50 mL). The combined organic layers were washed with brine (2×80 mL), dried over anhydrous Na₂SO₄. After filtration, the filtrate was concentrated under reduced pressure. The crude product was used in the next step directly without further purification. LCMS (ESI): mass calcd. for C₁₀H₁₅FNO₃, 213.1; m/z found, 214.3 [M+H]⁺.

### Preparation of methyl (2S)-2-[(tert-butoxycarbonyl)amino]-3-(3-fluoro-5-hydroxyphenyl) propanoate (I-13.2)

To a stirred mixture of methyl (S)-2-amino-3-(3-fluoro-5-hydroxyphenyl)propanoate (1.5 g, 7.035 mmol, 1 equiv) and NaHCO₃ (0.89 g, 10.553 mmol, 1.5 equiv) in dioxane/H₂O (15 mL/15 mL) under nitrogen atmosphere at room temperature was added Boc₂O (1.54 g, 7.035 mmol, 1 equiv) . The resulting mixture was stirred under nitrogen atmosphere at room temperature for 3 h., and then diluted with water. The resulting mixture was extracted with EtOAc (3×50 mL). The combined organic layers were washed with brine (2×80 mL), dried over anhydrous Na₂SO₄. After filtration, the filtrate was concentrated under reduced pressure. The residue was purified by reverse flash chromatography [with the following conditions: column, C18 silica gel; mobile phase, MeCN in Water (0.1% FA), 50% to 80% gradient in 15 min; detector, UV 254/220 nm]. This resulted in methyl (2S)-2-[(tert-butoxycarbonyl)amino]-3-(3-fluoro-5-hydroxyphenyl)propanoate as a white solid (750 mg, 34.02%). LCMS (ESI): mass calcd. for C₁₅H₂₀FNO₅, 313.1; m/z found, 312.2 [M−H]⁻.

### Preparation of 3-[(2S)-2-[(tert-butoxycarbonyl)amino]-3-methoxy-3-oxopropyl]-5-fluoro-phenoxymethylboronic acid (I-13.3)

To a stirred mixture of methyl (2S)-2-[(tert-butoxycarbonyl)amino]-3-(3-fluoro-5-hydroxyphenyl)propanoate (630 mg, 2.01 mmol, 1 equiv) and K₂CO₃ (1.39 g, 10.1 mmol, 5 equiv) in DMF (8 mL) under nitrogen atmosphere at room temperature was added 2-(iodomethyl)-4,4,5,5-tetramethyl-1,3,2-dioxaborolane (2.69.g, 10.055 mmol, 5 equiv) . The resulting mixture was stirred under nitrogen atmosphere at room temperature overnight. The resulting mixture was filtered, the filter cake was washed with MeOH (3×5 mL). The filtrate was concentrated under reduced pressure. The residue was purified by reversed-phase flash chromatography [with the following conditions: column, C18 silica gel; mobile phase, MeCN in Water (0.1% FA), 55% to 60% gradient in 10 min; detector, UV 254/220 nm]. This resulted in 3-[(2S)-2-[(tert-butoxycarbonyl)amino]-3-methoxy-3-oxopropyl]-5-fluorophenoxymethylboronic acid as a white solid (520 mg, 69.68%). LCMS (ESI): mass calcd. for C₁₆H₂₃BFNO₇, 371.2; m/z found, 272.1 [M+H−Boc]⁺.

### Preparation of (2S)-2-[(tert-butoxycarbonyl)amino]-3-{3-[(dihydroxyboranyl)methoxy]-5-fluorophenyl}propanoic acid(I-13.4)

To a stirred mixture of 3-[(2S)-2-[(tert-butoxycarbonyl)amino]-3-methoxy-3-oxopropyl]-5-fluorophenoxymethylboronic acid (888 mg, 2.392 mmol, 1 equiv) in THF/H₂O (16 mL/8 mL) under nitrogen atmosphere at room temperature was added LiOH·H₂O (301.16 mg, 7.177 mmol, 3.00 equiv) . The resulting mixture was stirred under nitrogen atmosphere at room temperature for 3 h, and then neutralized to “pH” 7 with 2N HC_{1.} The resulting mixture was concentrated under reduced pressure. The residue was purified by reversed-phase flash chromatography [with the following conditions: column, C18 silica gel; mobile phase, MeCN in Water (0.1% FA), 35% to 45% gradient in 10 min; detector, UV 254/220 nm]. This resulted in (2S)-2-[(tert-butoxycarbonyl)amino]-3-{3-[(dihydroxyboranyl)methoxy]-5-fluorophenyl}propanoic acid as a white solid (630 mg, 73.73%). LCMS (ESI): mass calcd. for C₁₅H₂₁BFNO₇, 357.1; m/z found, 356.1 [M−H]⁻.

### Preparation of (2S)-2-amino-3-[3-[(dihydroxyboranyl)methoxy]-5-fluorophenyl]propanoic acid (I-13)

To a stirred mixture of (2S)-2-[(tert-butoxycarbonyl)amino]-3-{3-[(dihydroxyboranyl)methoxy]-5-fluorophenyl}propanoic acid (210 mg, 0.588 mmol, 1 equiv) in EtOAc (2 mL) under nitrogen atmosphere at room temperature was added HCl (1 mL, 4 N in EtOAc) dropwise . The resulting mixture was stirred under nitrogen atmosphere at room temperature for 3 h, and then concentrated under vacuum. The residue was neutralized to “pH” 7 with NaOH (aq.). The mixture was purified by reversed-phase flash chromatography [with the following conditions: column, C18 silica gel; mobile phase, MeCN in Water (0.1% FA), 3% to 5% gradient in 8 min; detector, UV 254/220 nm]. The product was further purified by Prep-HPLC [with the following conditions (Column: XBridge Shield RP18 OBD Column, 30*150 mm, 5 μm; Mobile Phase A: Water (0.1% FA), Mobile Phase B: ACN; Flow rate: 50 mL/min; Gradient: 2% B to 18% B in 8 min, 18% B; Wave Length: 254/220 nm; RT1(min): 7.28)]. This resulted in (25)-2-amino-3-{3-[(dihydroxyboranyl)methoxy]-5-fluorophenyl}propanoic acid as a white solid (105 mg, 69.48%). LCMS (ESI): mass calcd. for C₁₀H₁₃BFNO₅, 257.1; m/z found, 258.2 [M+H]⁺. ¹H NMR (400 MHz, Deuterium Oxide) δ 6.69-6.61 (m, 2H), 6.59 (d, J=9.2 Hz, 1H), 3.91 (dd, J=7.9, 5.2 Hz, 1H), 3.72 (s, 2H), 3.16 (dd, J=14.5, 5.3 Hz, 1H), 3.00 (dd, J=14.5, 8.0 Hz, 1H).

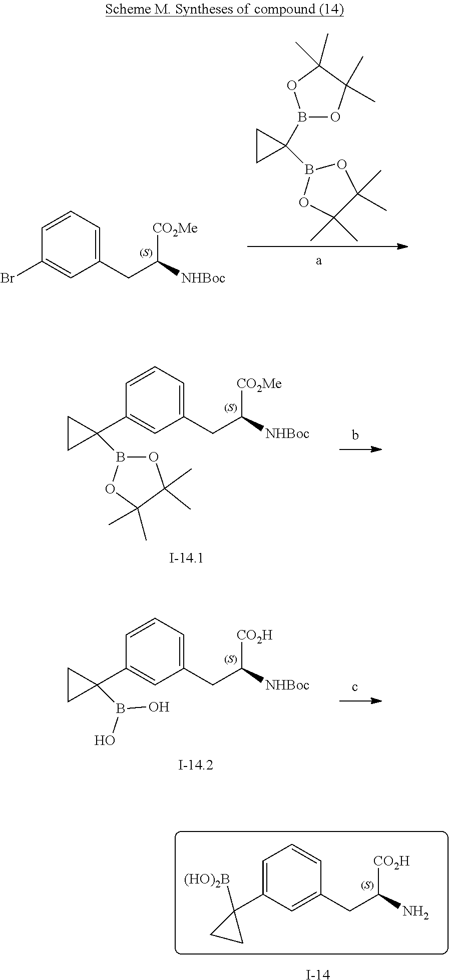

^{a}Reagents and Conditions: (a) CataCXium A Pd G3, Cs₂CO₃, dioxane/H2O, 100° C.; (b) LiOH·H₂O, THF, water, rt; (c) HCl, dioxane, rt;

### Preparation of methyl (2S)-2-[(tert-butoxycarbonyl)amino]-3-{3-[1-(4,4,5,5 -tetramethyl- 1,3,2-dioxaborolan-2-yl)cyclopropyl]phenyl}propanoate (I-14.1)

To a stirred mixture of methyl (2S)-3-(3-bromophenyl)-2-[(tert-butoxycarbonyl)amino]propanoate (731.06 mg, 2.041 mmol, 1.20 equiv) and 4,4,5,5-tetramethyl-2-[1-(4,4,5,5-tetramethyl-1,3,2-dioxaborolan-2-yl)cyclopropyl]-1,3,2-dioxaborolane (500 mg, 1.701 mmol, 1.00 equiv) in dioxane (16 mL)/H₂O (1.6 mL) was added CataCXium A Pd G3 (123.85 mg, 0.170 mmol, 0.1 equiv) and Cs2CO3 (1662.29 mg, 5.103 mmol, 3 equiv). The resulting mixture was purged with nitrogen for 1 min, and then stirred under nitrogen atmosphere at 100° C. for 5 h. The resulting mixture was concentrated under vacuum. The residue was purified by reverse flash chromatography [with the following conditions: column, C18 silica gel; mobile phase, MeCN in Water (0.1% FA), 60% to 80% gradient in 15 min; detector, UV 220 nm]. This resulted in methyl (2S)-2-[(tert-butoxycarbonyl)amino]-3-{3-[1-(4,4,5,5-tetramethyl-1,3,2-dioxaborolan-2-yl)cyclopropyl]phenyl}propanoate as a light yellow oil (240 mg, 31.69%). LCMS (ESI): mass calcd. for C₂₄H₃₆BNO₆, 445.3; m/z found, 446.3 [M+H]⁺.

### Preparation of (2S)-2-[(tert-butoxycarbonyl)amino]-3-{3-[1-(dihydroxyboranyl) cyclopropyl] phenyl}propanoic acid (1-14.2)

To a stirred mixture of methyl (2S)-2-[(tert-butoxycarbonyl)amino]-3-{3-[1-(4,4,5,5-tetramethyl-1,3,2-dioxaborolan-2-yl)cyclopropyl]phenyl}propanoate (240 mg, 0.539 mmol, 1 equiv) in H₂O (2.5 mL)/THF (2.5 mL) was added LiOH·H₂O (67.84 mg, 1.617 mmol, 3 equiv). The resulting mixture was stirred at room temperature for 3 h. The mixture was acidified to “pH” 7 with 2 M HCl (aq.) and concentrated under vacuum. The residue was purified by reverse flash chromatography [with the following conditions: column, C18 silica gel; mobile phase, MeCN in Water (0.1% FA), 40% to 60% gradient in 10 min; detector, UV 220 nm]. This resulted in (2S)-2-[(tert-butoxycarbonyl)amino]-3-{3-[1-(dihydroxyboranyl)cyclopropyl]phenyl}propanoic acid as a colorless oil (120 mg, 63.77%). LCMS (ESI): mass calcd. for C₁₇H₂₄BNO₆, 349.2; m/z found, 250.1 [M−Boc+H]⁺

### Preparation of (2S)-2-amino-3-{3-[1-(dihydroxyboranyl)cyclopropyl] phenyl}propanoic acid (I-14)

To a stirred mixture of (2S)-2-[(tert-butoxycarbonyl)amino]-3-{3-[1-(dihydroxyboranyl) cyclopropyl] phenyl}propanoic acid (150 mg, 0.430 mmol, 1 equiv) in dioxane (2.0 mL) was added a 1,4-dioxane solution (4 mL) of HCl (0.7 mL) in dropwise. The resulting mixture was stirred at room temperature for 3 h, and then concentrated under vacuum. The crude product was purified by Prep-HPLC [with the following conditions (Column: XBridge Prep Phenyl OBD Column, 19*150 mm, 5 μm; Mobile Phase A: Water (0.1% FA), Mobile Phase B: MeOH-HPLC; Flow rate: 25 mL/min; Gradient: 17% B to 42% B in 7 min, 42% B; Wave Length: 220 nm; RT1(min): 5.11)] to afford (2S)-2-amino-3-{3-[1-(dihydroxyboranyl)cyclopropyl] phenyl}propanoic acid as a white solid (27 mg, 25.24%). LCMS (ESI): mass calcd. for C₁₂H₁₆BNO₄, 249.1; m/z found, 250.2 [M+H]⁺. ¹H NMR (400 MHz, Deuterium Oxide) δ 7.27-7.18 (m, 2H), 7.16 (s, 1H), 7.04 (d, J=7.0 Hz, 1H), 3.89 (dd, J=8.0, 5.2 Hz, 1H), 3.17 (dd, J=14.5, 5.2 Hz, 1H), 2.98 (dd, J=14.5, 8.3 Hz, 1H), 1.05-0.97 (m, 2H), 0.87-0.80 (m, 2H).

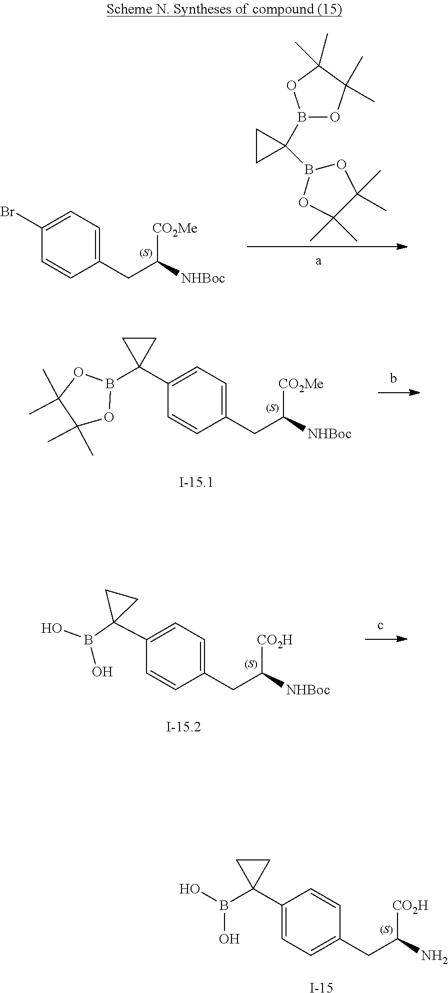

Reagents and Conditions: (a) CataCXium A Pd G3, Cs₂CO₃, dioxane/H20, 100° C.; (b) LiOH·H₂O, THF, water, rt; (c) HCl, dioxane, rt.

### Preparation of methyl (2S)-2-[(tert-butoxycarbonyl)amino]-3-{3-[1-(4,4,5,5-tetramethyl-1,3,2-dioxaborolan-2-yl)cyclopropyl]phenyl}propanoate (I-15.1)

To a stirred mixture mixture of methyl (2S)-3-(4-bromophenyl)-2-[(tert-butoxycarbonyl) amino]propanoate (292.42 mg, 0.816 mmol, 1.2 equiv) and 4,4,5,5-tetramethyl-2-[1-(4,4,5,5-tetramethyl-1,3,2-dioxaborolan-2-yl)cyclopropyl]-1,3,2-dioxaborolane (200 mg, 0.680 mmol, 1.00 equiv) in dioxane (6 mL)/H₂O (0.6 mL) was added CataCXium A Pd G3 (74.31 mg, 0.102 mmol, 0.15 equiv) and Cs₂CO₃ (54.57 mg, 0.168 mmol, 3 equiv). The resulting mixture was purged with nitrogen for 1 min, and then stirred under nitrogen atmosphere at 80° Cfor 5 h. The resulting mixture was concentrated under vacuum. The residue was purified by reverse flash chromatography [with the following conditions: column, C18 silica gel; mobile phase, MeCN in Water (0.1% FA), 60% to 80% gradient in 20 min; detector, UV 220 nm]. This resulted in methyl (2S)-2-[(tert-butoxycarbonyl)amino]-3-{4-[1-(4,4,5,5-tetramethyl-1,3 ,2-dioxaborolan-2-yl)cyclopropyl]phenyl }propanoate as a light yellow oil (110 mg, 36.31%). LCMS (ESI): mass calcd. for C₂₄H₃₆BNO₆, 445.3; m/z found, 468.2 [M+Na]⁺.

### Preparation of (2S)-2-[(tert-butoxycarbonyl)amino]-3-{4-[1-(dihydroxyboranyl) cyclopropyl] phenyl}propanoic acid (I-15.2)

To a stirred mixture of methyl (2S)-2-[(tert-butoxycarbonyl)amino]-3-{4-[1-(4,4,5,5-tetramethyl-1,3,2-dioxaborolan-2-yl)cyclopropyl]phenyl}propanoate (100 mg, 0.225 mmol, 1 equiv) in H₂O (1.0 mL)/THF (1.0 mL) was added LiOH·H₂O (28.26 mg, 0.675 mmol, 3 equiv). The resulting mixture was stirred at room temperature for 3 h. The mixture was acidified to “pH” 7 with 2 M HCl (aq.) and then concentrated under vacuum. The residue was purified by reversed-phase flash chromatography [with the following conditions: column, C18 silica gel; mobile phase, MeCN in Water (0.1% FA), 40% to 60% gradient in 15 min; detector, UV 220 nm]. This resulted in (2S)-2-[(tert-butoxycarbonyl)amino]-3-{4-[1-(dihydroxyboranyl)cyclopropyl]phenyl}propanoic acid as a colorless oil (70 mg, 89.28%). LCMS (ESI): mass calcd. for C₁₇H₂₄BNO₆, 349.2; m/z found, 250.1 [M−Boc+H]⁺.

### Preparation of (2S)-2-amino-3-{4-[1-(dihydroxyboranyl)cyclopropyl]phenyl}propanoic acid (I-15)

To a stirred mixture of (2S)-2-[(tert-butoxycarbonyl)amino]-3-{4-[1-(dihydroxyboranyl) cyclopropyl] phenyl}propanoic acid (70 mg, 0.200 mmol, 1 equiv) in dioxane (1.2 mL) was added a 1,4-dioxane solution (4M) of HCl (0.40 mL) dropwise. The resulting mixture was stirred at room temperature for 3 h, and then concentrated under vacuum. The crude product was purified by Prep-HPLC [with the following conditions (Column: Xselect CSH C18 OBD Column 30*150 mm 5 μm; Mobile Phase A: Water (0.1%FA), Mobile Phase B: ACN; Flow rate: 60 mL/min; Gradient: 7% B to 19% B in 7 min, 19% B; Wave Length: 220 nm; RT1 (min): 6.78)] to afford (2S)-2-amino-3-{4-[1-(dihydroxyboranyl)cyclopropyl]phenyl}propanoic acid as a white solid (31 mg, 62.09%). LCMS (ESI): mass calcd. for C₁₂H₁₆BNO₄, 249.1; m/z found, 250.1 [M+H]⁺. ¹H NMR (400 MHz, Deuterium Oxide) δ 7.24 (d, J=7.9 Hz, 2H), 7.12 (d, J=7.9 Hz, 2H), 3.91-3.81 (m, 1H), 3.14 (dd, J=14.5, 5.3 Hz, 1H), 2.99 (dd, J=14.5, 7.8 Hz, 1H), 1.05-0.92 (m, 2H), 0.88-0.74 (m, 2H).

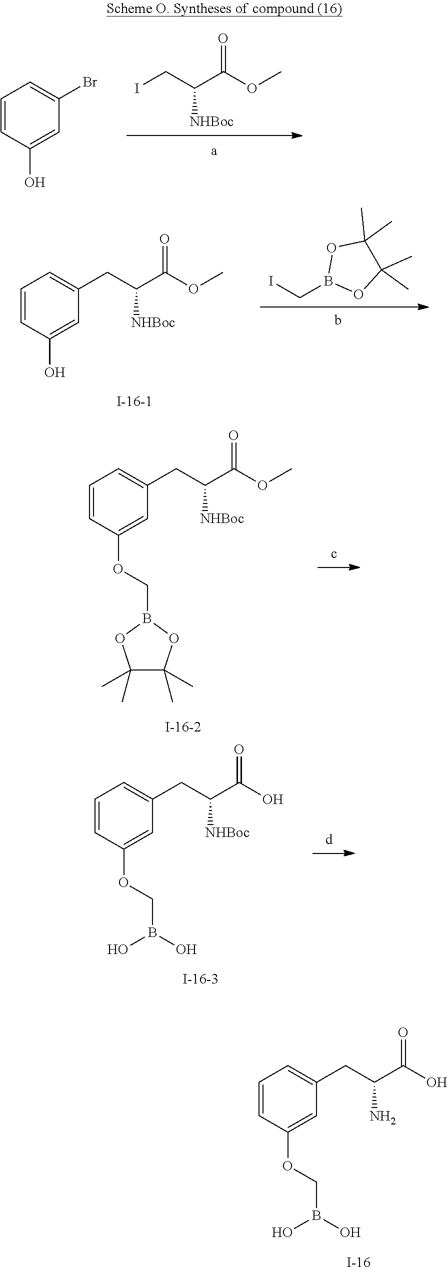

Reagents and Conditions: (a) Zn, 12, Pd2(dba)3, S-Phos, DMF, 45° C.; (b) K₂CO₃, DMF, rt; (c) LiOH. H₂O, THF, H₂O, rt; (d) HCl, dioxane, rt.

### Preparation of methyl (2R)-2-[(tert-butoxycarbonyl)amino]-3-(3-hydroxyphenyl)propanoate (I-16-1)

A mixture of Zn (3.4 g, 52.02 mmol, 3 equiv) and I₂ (0.22 g, 1.73 mmol, 0.1 equiv) in DMF (30 mL) was stirred under nitrogen atmosphere at room temperature for 5 min. To the above mixture was added a solution of methyl (2S)-2-[(tert-butoxycarbonyl)amino]-3-iodopropanoate (6.85 g, 20.81 mmol, 1.2 equiv) in DMF (30 mL) slowly, followed by the addition of I₂ (0.22 g, 1.73 mmol, 0.1 equiv). The resulting mixture was stirred under nitrogen atmosphere at room temperature for 30 min. Then, to the mixture was added a suspension of 3-bromophenol (3 g, 17.34 mmol, 1 equiv), S-Phos (0.71 g, 1.73 mmol, 0.1 equiv) and Pd2(dba)₃ (0.79 g, 0.86 mmol, 0.05 equiv) in DMF (10 mL). The reaction was stirred under nitrogen atmosphere at 45° C. overnight. The reaction was quenched with water (20 mL), diluted with EA (80 mL), and filtered through Celite. The filtrate was washed with brine (3×20 mL), dried over anhydrous Na₂SO₄, filtered, and concentrated. The residue was purified by reversed-phase flash chromatography [with the following conditions: column, C18 silica gel; mobile phase, MeCN in water (0.1% FA), 40% to 60% gradient in 20 min; detector, UV 254 nm] to provide the title product as a yellow oil (2.7g, 52.7%). LCMS (ESI): mass calcd. for C₁₅H₂₁NO₅, 295.1; m/z found, 296.1 [M+H]⁺.

### Preparation of methyl (2R)-2-[(tert-butoxycarbonyl)amino]-3-{3-[(4,4,5,5-tetrarnethyl-1,3,2-dioxaborolan-2-yl)methoxy]phenyl}propanoate (I-16-2)

To a stirred mixture of methyl (2R)-2-[(tert-butoxycarbonyl)amino]-3-(3-hydroxyphenyl) propanoate (2.7 g, 9.14 mmol, 1 equiv) and K₂CO₃ (1.9 g, 13.71 mmol, 1.5 equiv) in DMF (20 mL) was added 2-(iodomethyl)-4,4,5,5-tetramethyl-1,3,2-dioxaborolane (2.9 g, 10.97 mmol, 1.2 equiv) slowly. The resulting mixture was stirred under nitrogen atmosphere at room temperature overnight. The reaction was quenched with water (10 mL), and extracted with EtOAc (3×30 mL). The combined organic layers were washed with brine (2×20 mL), dried over anhydrous Na₂SO₄, filtered, concentrated under reduced pressure. The residue was purified by reversed-phase flash chromatography [with the following conditions: column, C18 silica gel; mobile phase, MeCN in water (10 mmol/L NH4HCO3), 10% to 40% gradient in 60 min; detector, UV 254 nm] to afford the title product as a brown oil (675 mg, 17.0%). LCMS (ESI): mass calcd. for C₂₄H₃₅BFNO₆, 435.2; m/z found, 434.2 [M−H]⁻.

### Preparation of (2R)-2-[(tert-butoxycarbonyl)amino]-3-{3-[(dihydroxyboranyl)methoxy] phenyl}propanoic acid (I-16-3)

To a stirred mixture of methyl (2R)-2-[(tert-butoxycarbonyl)aminol-3-{3-[(4,4,5,5-tetramethyl-1,3,2-dioxaborolan-2-yl)methoxy]phenyl}propanoate (391 mg, 0.90 mmol, 1 equiv) in THF (8 mL)/H₂O (4 mL) was added LiOH·H₂O (75.6 mg, 1.80 mmol, 2 equiv). The resulting mixture was stirred at room temperature for 3 h. Then the mixture was acidified to pH 5 with 2N HCl (aq.), and extracted with EA (3×20 mL). The combined organic layers were washed with brine (20 mL), dried over anhydrous Na₂SO₄, filtered, concentrated under reduced pressure. The residue was purified by reversed-phase flash chromatography [with the following conditions: column, C18 silica gel; mobile phase, MeCN in water (0.1% FA), 10% to 50% gradient in 30 min; detector, UV 220 nm] to afford the title product as a light yellow solid (215 mg, 70.6%). LCMS (ESI): mass calcd. for C₁₅H₂₂BNO₇, 339.2; m/z found, 337.8 [M−H]⁻.

### Preparation of (2R)-2-amino-3-{3-[(dihydroxyboranyl)methoxy]phenyl}propanoic acid (16)

To a stirred solution of (2R)-2-[(tert-butoxycarbonyl)amino]-3-{3-[(dihydroxyboranyl) methoxy]phenyl}propanoic acid (200 mg, 0.59 mmol, 1 equiv) in dioxane (2 mL) was added HC₁ (2 mL, 4 mol/L in dioxane) slowly. The resulting mixture was stirred under nitrogen atmosphere at room temperature for 2 h. Then the mixture was concentrated under reduced pressure. The residue was purified by Prep-HPLC [with the following conditions: Column: XBridge Prep Amide OBD Column, 19*150 mm, 5 μm; Mobile Phase A: Water (0.05%TFA), Mobile Phase B: ACN; Flow rate: 60 mL/min mL/min; Gradient: 95% B to 83% B in 10 min; Wave Length: 254nm/220nm nm; RT1(min): 8.32] to afford the title product as a white solid (57.3 mg, 40.5%). LCMS (ESI): mass calcd. for C₁₀H₁₄BNO₅, 239.10; m/z found, 240.10 [M+H]⁺; ¹H NMR (400 MHz, Deuterium Oxide) δ 7.33-7.21 (m, 1H), 6.93-6.86 (m, 1H), 6.86-6.79 (m, 2H), 4.29-4.16 (m, 1H), 3.80-3.64 (m, 2H), 3.32-3.16 (m, 1H), 3.16-3.01 (m, 1H).

### Example 2. Cell Uptake Assays

### Cell Culture

### 1.1 Thawing Method

1.1.1 15 mL of cell culture medium was placed into a T225 flask.
1.1.2 The flask was placed in a humidified 37° C., 5% CO₂ incubator for 15 minutes to allow medium to equilibrate to the proper pH and temperature.
1.1.3 The vial was removed from liquid nitrogen and thawed rapidly by placing at 37° C. in a water bath with gentle agitation for 1-2 minutes and then decontaminated by wiping with 70% ethanol before opening in a Class II biological safety cabinet.
1.1.4 The vial contents were transferred dropwise into 10 mL of cell culture medium in a sterile 15 mL conical tube.
1.1.5 The cells were centrifuged at 1,300 rpm for 5 minutes.
1.1.6 The supernatant was aspirated and re-suspended the cells and transferred the cells into T225 flask containing cell culture medium.

### 1.2 Propagation Method

1.2.1 Culture medium were renewed every 2-3 days.
1.2.2 Keep the flask in a humidified 37° C., 5% CO₂ incubator

### Assay Procedures

### 2.1 Cell Seeding

Cells were harvested and diluted in culture medium to the designated concentration. Then the cells were cultured in T25 flasks, one T25 flask for one sample.
   2.2 Formulation of test compounds
2.2.1 L-Boronophenylalanine (L-BPA, 4-boron-L-phenylalanine, Purity: 95%, Chemical formula: C₉H12BNO4, MW: 209.01, CAS: 76410-58-7) and fructose were dissolved in PBS at a molar ratio of 1:5, and then a 1.27 M ratio of 1 N NaOH is added. The mixture was stirred until L-BPA is completely dissolved, and the pH value was titrated to 7.2-7.4 with 1 N HC_{1.}
2.2.2 For test compounds other than BPA, stock solutions were prepared of each compound in DMSO or fructose formulation at a concentration of 20 mM (such that final % DMSO in the cellular assay medium=0.5%)
   2.3 Compound treatment
2.3.1 The cells were seeded overnight (12-18 hours) to allow the cells to adhere well.
2.3.2 The stock solution of each compound was added to each T25 flask such that the final concentration for each compound is 100 uM (0.1 mM).
2.3.3 The flasks were placed in a 37° C., 5% CO₂ incubator for 1 hour, 4 hours and 24 hours.
2.3.4 After treatment for 1 hour, 4 hour, and 24 hours, the cells were collected by using 0.25% Trypsin with 0.53 mM EDTA.
2.3.5 Centrifuged at 1,300 rpm for 5 min to collect cell pellets.

### Results

The compounds disclosed herein were selectively taken up by the representative human cancer cell lines SAS (head & neck cancer), U87-MG (glioblastoma) and B6 (melanoma) relative to a representative normal human cell line (NIH-3T3). Compounds 1-3, 10-11, and 13-15, in addition to showing selective partitioning into cancer cells relative to normal cells, showed increased uptake in cancer cells relative to BPA.

TABLE 1
Cell uptake of compound 1 as compared to BPA

| 1 | | | | | BPA | | | | |
|---|---|---|---|---|---|---|---|---|---|
| Time | Mean (ng/10⁶ cell) | | | | Time | Mean (ng/10⁶ cell) | | | |
| (h) | SAS | B16F10 | NIH-3T3 | U87-MG | (h) | SAS | B16F10 | NIH-3T3 | U87-MG |
| 1.00 | [symbol] | 112 | 127 | 227 | 1.00 | 294 | 1 [symbol] 8 | 91.4 | 101 |
| 4.00 | 755 | 232 | 194 | 233 | 4.00 | 259 | 120 | 109 | 85.2 |
| 24.0 | 828 | 517 | 133 | 336 | 24.0 | 962 | 257 | 70.1 | 136 |

[symbol]

indicates data missing or illegible when filed

TABLE 2a
Cell uptake of compound 2 as compared to BPA

| 2 | | | | | BPA | | | | |
|---|---|---|---|---|---|---|---|---|---|
| Time | Mean (ng/10⁶ cell) | | | | Time | Mean (ng/10⁶ cell) | | | |
| (h) | SAS | B16F10 | NIH-3T3 | U87-MG | (h) | SAS | B16F10 | NIH-3T3 | U87-MG |
| 1.00 | 1125 | 151 | [symbol] .5 | 123 | 1.00 | 294 | 108 | 91.4 | 101 |
| 4.00 | 1590 | 333 | 20 [symbol] | 40 [symbol] | 4.00 | 259 | 120 | 109 | 85.2 |
| 24.0 | 1595 | 881 | 205 | 626 | 24.0 | 962 | 2 [symbol] 7 | 70.1 | 136 |

[symbol]

indicates data missing or illegible when filed

TABLE 3
Cell uptake of compound 3 as compared to BPA

| 3 | | | | | BPA | | | | |
|---|---|---|---|---|---|---|---|---|---|
| Time | Mean (ng/10⁶ cell) | | | | Time | Mean (ng/10⁶ cell) | | | |
| (h) | SAS | B16F10 | NIH-3T3 | U87-MG | (h) | SAS | B16F10 | NIH-3T3 | U87-MG |
| 1.00 | 534 | 145 | 132 | 197 | 1.00 | 294 | 108 | 91.4 | 101 |
| 4.00 | 499 | 222 | 138 | 211 | 4.00 | 259 | 120 | 109 | 85.2 |
| 24.0 | NA | 117 | 7 [symbol] | 183 | 24.0 | 962 | 257 | 70.1 | 136 |

[symbol]

indicates data missing or illegible when filed

TABLE 4
Cell uptake of compound 4 as compared to BPA

| 4 | | | | | BPA | | | | |
|---|---|---|---|---|---|---|---|---|---|
| Time | Mean (ng/10⁶ cell) | | | | Time | Mean (ng/10⁶ cell) | | | |
| (h) | SAS | B16F10 | NIH-3T3 | U87-MG | (h) | SAS | B16F10 | NIH-3T3 | U87-MG |
| 1.00 | 445 | 46.1 | 27.1 | 42.8 | 1.00 | 294 | 10 [symbol] | 91.4 | 101 |
| 4.00 | 495 | 95.6 | 58.6 | 113 | 4.00 | 259 | 120 | 109 | 85.2 |
| 24.0 | 997 | 101 | 66.6 | 191 | 24.0 | 9 [symbol] 2 | 257 | 70.1 | 138 |

[symbol]

indicates data missing or illegible when filed

TABLE 5
Cell uptake of compound 5 as compared to BPA

| 5 | | | | | BPA | | | | |
|---|---|---|---|---|---|---|---|---|---|
| Time | Mean (ng/10⁶ cell) | | | | Time | Mean (ng/10⁶ cell) | | | |
| (h) | SAS | B16F10 | NIH-3T3 | U87-MG | (h) | SAS | B16F10 | NIH-3T3 | U87-MG |
| 1.00 | 1 [symbol] 3 | 12 [symbol] | 171 | 1 [symbol] | 1.00 | 2 [symbol] 7 | 126 | 105 | 156 |
| 4.00 | 225 | 157 | 77.2 | 1 [symbol] | 4.00 | 185 | 1 [symbol] | 9 [symbol] 2 | 1 [symbol] 7 |
| 24.0 | 214 | 262 | [symbol] 0.3 | 12 [symbol] | 24.0 | 1 [symbol] 3 | 332 | 9 [symbol] | 156 |

[symbol]

indicates data missing or illegible when filed

TABLE 6
Cell uptake of compound 6 as compared to BPA

| 6 | | | | | BPA | | | | |
|---|---|---|---|---|---|---|---|---|---|
| Time | Mean (ng/10⁶ cell) | | | | Time | Mean (ng/10⁶ cell) | | | |
| (h) | SAS | B16F10 | NIH-3T3 | U87-MG | (h) | SAS | B16F10 | NIH-3T3 | U87-MG |
| 1.00 | 152 | [symbol] | 53.4 | 160 | 1.00 | 297 | 126 | 105 | 156 |
| 4.00 | 106 | 92.3 | 23.2 | 137 | 4.00 | 185 | 188 | 98.2 | 167 |
| 24.0 | 46.0 | 2 [symbol] .0 | NA | 1 [symbol] 7 | 24.0 | 193 | 332 | 92 [symbol] | 156 |

[symbol]

indicates data missing or illegible when filed

TABLE 7
Cell uptake of compound 8 as compared to BPA

| 8 | | | | | BPA | | | | |
|---|---|---|---|---|---|---|---|---|---|
| Time | Mean (ng/10⁶ cell) | | | | Time | Mean (ng/10⁶ cell) | | | |
| (h) | SAS | B16F10 | NIH-3T3 | U87-MG | (h) | SAS | B16F10 | NIH-3T3 | U87-MG |
| 1.00 | 44.6 | 26.9 | 14.7 | 16.7 | 1.00 | 163 | 102 | 69.7 | 9 [symbol] .6 |
| 4.00 | 66.7 | 31.4 | 26.4 | 31.1 | 4.00 | 157 | 143 | 98.0 | 127 |
| 24.0 | 359 | 38. [symbol] | 3 [symbol] .4 | 16.3 | 24.0 | 328 | 144 | 38.2 | 94.6 |

[symbol]

indicates data missing or illegible when filed

TABLE 8
Cell uptake of compound 9 as compared to BPA

| 9 | | | | | BPA | | | | |
|---|---|---|---|---|---|---|---|---|---|
| Time | Mean (ng/10⁶ cell) | | | | Time | Mean (ng/10⁶ cell) | | | |
| (h) | SAS | B16F10 | NIH-3T3 | U87-MG | (h) | SAS | B16F10 | NIH-3T3 | U87-MG |
| 1.00 | 129 | 67 [symbol] | 34.4 | 90.1 | 1.00 | 163 | 102 | 69.7 | 9 [symbol] |
| 4.00 | 103 | 66.7 | 43.0 | 1 [symbol] 7 | 4.00 | 157 | 143 | 9 [symbol] | 127 |
| 24.0 | 211 | 119 | 37.2 | 74.6 | 24.0 | 328 | 144 | 38.2 | 94.6 |

[symbol]

indicates data missing or illegible when filed

TABLE 9
Cell uptake of compound 10 as compared to BPA

| 10 | | | | | BPA | | | | |
|---|---|---|---|---|---|---|---|---|---|
| Time | Mean (ng/10⁶ cell) | | | | Time | Mean (ng/10⁶ cell) | | | |
| (h) | SAS | B16F10 | NIH-3T3 | U87-MG | (h) | SAS | B16F10 | NIH-3T3 | U87-MG |
| 1.00 | 1285 | 23 [symbol] | 81.9 | 295 | 1.00 | 782 | 252 | 97. [symbol] | 197 |
| 4.00 | 1850 | 442 | 204 | 673 | 4.00 | [symbol] 11 | 234 | 10 [symbol] | 317 |
| 24.0 | 14 [symbol] 0 | 814 | 12 [symbol] | 681 | 24.0 | 1345 | 267 | 75.9 | 172 |

[symbol]

indicates data missing or illegible when filed

TABLE 10
Cell uptake of compound 11 as compared to BPA

| 11 | | | | | BPA | | | | |
|---|---|---|---|---|---|---|---|---|---|
| Time | Mean (ng/10⁶ cell) | | | | Time | Mean (ng/10⁶ cell) | | | |
| (h) | SAS | B16F10 | NIH-3T3 | U87-MG | (h) | SAS | B16F10 | NIH-3T3 | U87-MG |
| 1.00 | 529 | 50.6 | 32.4 | 113 | 1.00 | 1 [symbol] 8 | 148 | 110 | 410 |
| 4.00 | 454 | 121 | 92.8 | 322 | 4.00 | 3 [symbol] 0 | 227 | 129 | 345 |
| 24.0 | 999 | 202 | 122 | 309 | 24.0 | 49 [symbol] | 315 | 133 | 178 |

[symbol]

indicates data missing or illegible when filed

TABLE 11
Cell uptake of compound 14 as compared to BPA

| 14 | | | | | BPA | | | | |
|---|---|---|---|---|---|---|---|---|---|
| Time | Mean (ng/10⁶ cell) | | | | Time | Mean (ng/10⁶ cell) | | | |
| (h) | SAS | B16F10 | NIH-3T3 | U87-MG | (h) | SAS | B16F10 | NIH-3T3 | U87-MG |
| 1.00 | 1 [symbol] 0 | 120 [symbol] | [symbol] | 15 [symbol] | 1.00 | 782 | 252 | 97.8 | 197 |
| 4.00 | 44 [symbol] | 12 [symbol] | 444 | 117 [symbol] | 4.00 | 811 | 234 | 106 | 317 |
| 24.0 | 4 [symbol] 0 | 2 [symbol] | 10 [symbol] | 24 [symbol] | 24.0 | 1345 | 267 | 75.9 | 172 |

[symbol]

indicates data missing or illegible when filed

TABLE 12
Cell uptake of compound 15 as compared to BPA

| 15 | | | | | BPA | | | | |
|---|---|---|---|---|---|---|---|---|---|
| Time | Mean (ng/10⁶ cell) | | | | Time | Mean (ng/10⁶ cell) | | | |
| (h) | SAS | B16F10 | NIH-3T3 | U87-MG | (h) | SAS | B16F10 | NIH-3T3 | U87-MG |
| 1.00 | 491 | 193 | 223 | 225 | 1.00 | 782 | 252 | 97.8 | 197 |
| 4.00 | 283 | 2 [symbol] 0 | 185 | 337 | 4.00 | [symbol] 11 | 234 | 10 [symbol] | 317 |
| 24.0 | 419 | 458 | 169 | 269 | 24.0 | 1345 | 267 | 75.9 | 172 |

[symbol]

indicates data missing or illegible when filed

TABLE 13
Cell uptake of compound 13 as compared to BPA

| 13 | | | | | BPA | | | | |
|---|---|---|---|---|---|---|---|---|---|
| Time | Mean (ng/10⁶ cell) | | | | Time | Mean (ng/10⁶ cell) | | | |
| (h) | SAS | B16F10 | NIH-3T3 | U87-MG | (h) | SAS | B16F10 | NIH-3T3 | U87-MG |
| 1 | 903 | 180 | 183 | 521 | 1 | 286 | 135 | 118 | 209 |
| 4 | 1012 | 310 | 222 | 828 | 4 | 225 | 261 | 112 | 247 |
| 24 | 1077 | 304 | 168 | 735 | 24 | 556 | 394 | 98.8 | 293 |

### INCORPORATION BY REFERENCE

All of the U.S. patents and U.S. and PCT patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A compound of Formula (I): 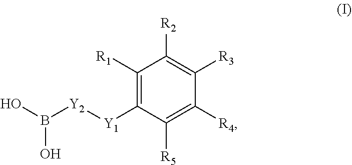
wherein
Y₁ is absent or —O—;
Y₂ is optionally substituted —alkylene—;
R₁, R₂, and Rs are each independently selected from —H and halo;
R₃ is selected from —H, halo, and —X₁-X₂;
R₄ is selected from —H, halo, and —X₁-X₂;
X₁ is —alkylene—;
X₂ is —C(H)(NH₂)CO₂H; and
the compound is racemic, enriched in one enantiomer, or a single enantiomer;
provided that the compound comprises one and only one occurrence of —X₁-X₂;
or a pharmaceutically acceptable salt thereof.

2. The compound of claim 1 having the structure of Formula (IA): 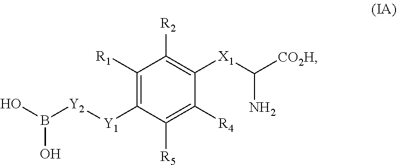
wherein R₄ is selected from —H and halo.

3. The compound of claim 2, wherein each of R₁, R₂, R₄, and R₅ is —H; or one of R₁, R₂, R₄, and R₅ is halo, and each of the remainder of R₁, R₂, R₄, and R₅ is —H.

4. (canceled)

5. (canceled)

6. The compound of claim 2 having the structure selected from: 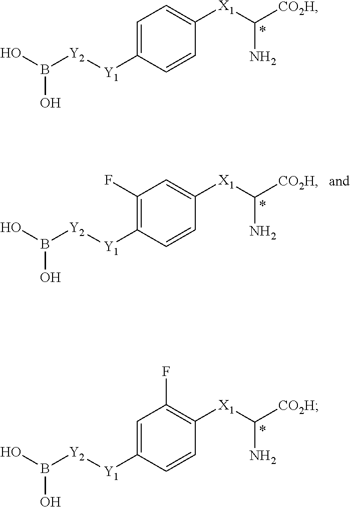
wherein * indicates a chiral carbon with an absolute configuration of (S) or (R); and the compound is not racemic.

7. The compound of claim 6, wherein the absolute configuration of the chiral carbon is (S).

8. The compound of claim 1 having the structure of Formula (TB): 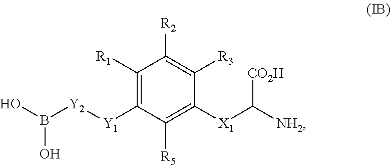
wherein R₃ is selected from —H and halo.

9. The compound of claim 8, wherein each of R₁, R₂, R₃, and R₅ is —H; or one of R₁, R₂, R₄, and R₅ is halo, and each of the remainder of R₁, R₂, R₄, and R₅ is —H.

10. (canceled)

11. (canceled)

12. The compound of claim 8 having the structure selected from: 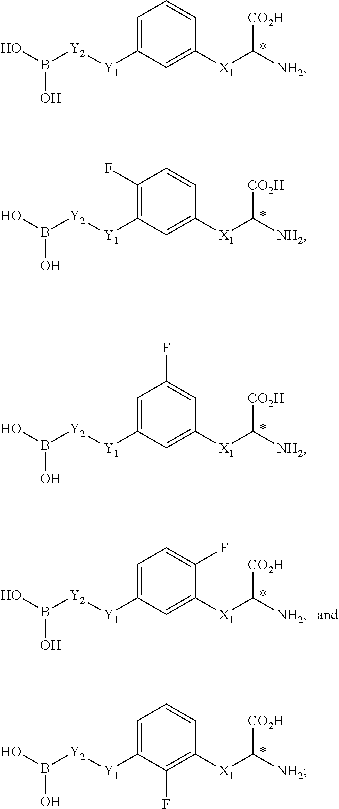
wherein * indicates a chiral carbon with an absolute configuration of (S) or (R); and the compound is not racemic.

13. The compound of claim 12, wherein the absolute configuration of the chiral carbon is (S).

14. The compound of claim 1, wherein X₁ is —(C₁-C₄)alkylene—.

15. (canceled)

16. The compound of claim 1, wherein Y₁ is —O—.

17. The compound of claim 1, wherein Y₁ is absent.

18. The compound of claim 16, wherein Y₂ is unsubstituted —(C₁-C₄)alkylene—.

19. (canceled)

20. (canceled)

21. The compound of claim [[20]]16, wherein Y₂ is seleted from —C(Y₃)(Y₄)— and —C(Y₃)(Y₄)CH₂—; and
Y₃ and Y₄ are each independently selected from —H, halo, alkyl, and heteroalkyl, provided that at least one of Y₃ and Y₄ is not —H; or Y₃ and Y₄ taken together with the carbon to which they are bonded form a cycloalkyl, cycloheteroalkyl, spiro cycloalkyl or spiro cycloheteroalkyl.

22. (canceled)

23. The compound of claim 17, wherein Y₂ is unsubstituted —(C₁-C₄)alkylene—.

24. (canceled)

25. (canceled)

26. The compound of claim 17, wherein Y₂ is seleted from —C(Y₃)(Y₄)— and —C(Y₃)(Y₄)CH₂—; and
Y₃ and Y₄ are each independently selected from —H, halo, alkyl, and heteroalkyl, provided that at least one of Y₃ and Y₄ is not —H; or
Y₃ and Y₄ taken together with the carbon to which they are bonded form a cycloalkyl, cycloheteroalkyl, spiro cycloalkyl or spiro cycloheteroalkyl.

27. (canceled)

28. The compound of claim 1 having the structure selected from: 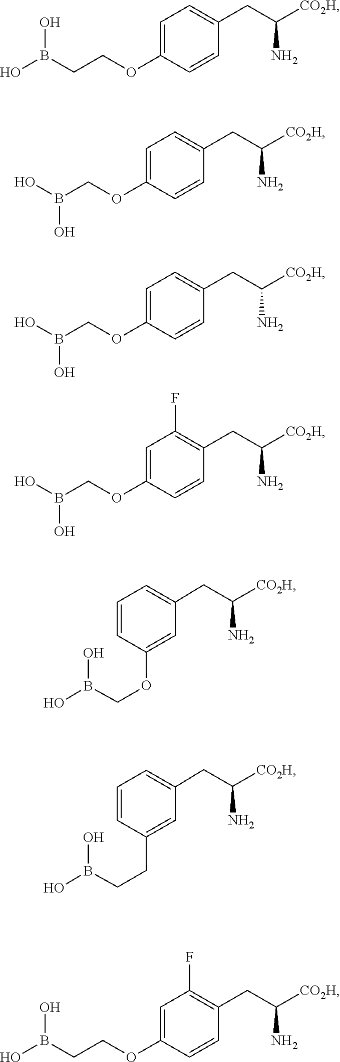 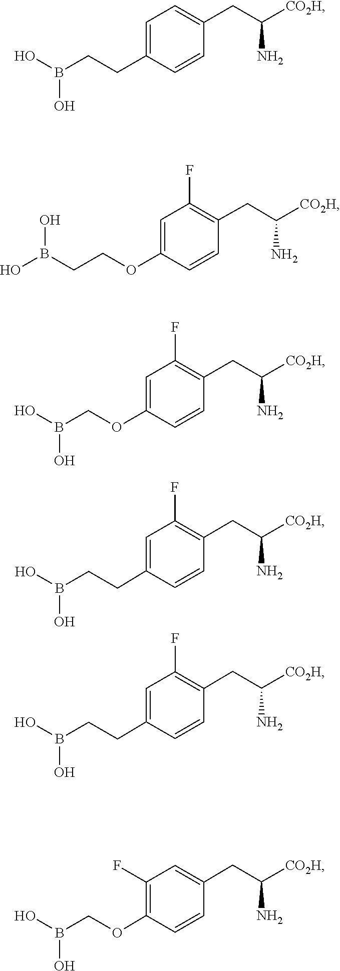 [SMILES: N[C@@H](Cc1ccc(CCB(O)O)cc1)C(=O)O ; N[C@H](Cc1ccc(OCCB(O)O)cc1F)C(=O)O ; N[C@@H](Cc1ccc(OCB(O)O)cc1F)C(=O)O ; N[C@@H](Cc1ccc(CCB(O)O)cc1F)C(=O)O ; N[C@H](Cc1ccc(CCB(O)O)cc1F)C(=O)O ; N[C@@H](Cc1ccc(OCB(O)O)c(F)c1)C(=O)O] 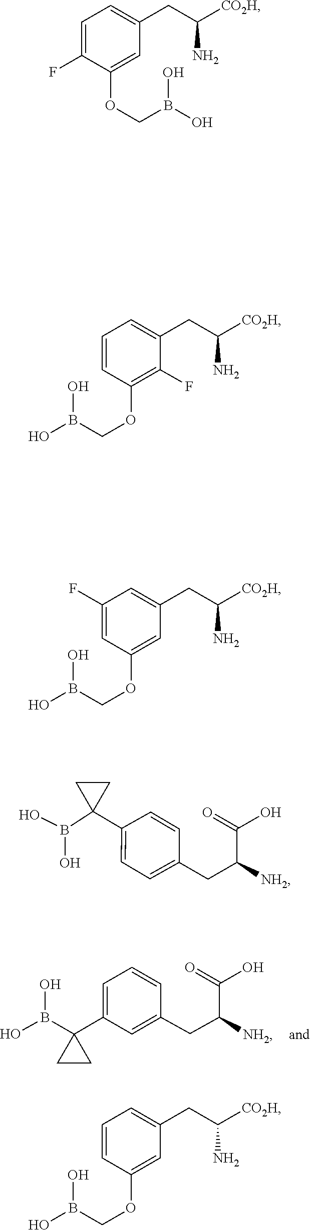
or a pharmaceutically acceptable salt thereof.

29. (canceled)

30. The compound of claim 1, wherein the boron atom in the compound is ¹⁰B.

31. A pharmaceutical composition, comprising a compound of claim 1; and a pharmaceutical acceptable excipient.

32. (canceled)

33. (canceled)

34. (canceled)

35. A method of treating cancer, comprising:
i) administering to a subject in need thereof a compound of claim 1, wherein the compound accumulates in a plurality of cancer cells in the subject; and
ii) irradiating the plurality of cancer cells with neutrons.

36.-46. (canceled)

47. The method of claim 35, wherein the cancer is selected from head and neck cancer, glioblastoma, melanoma, sarcoma, breast cancer, meningioma, lung cancer, mesothelioma, hepatocellular carcinoma, and extramammary Paget disease.

48. (canceled)
